SEQUENCE LISTING (28 sequences)

SEQ ID NO: 1-3 - skipped (3 entries)

SEQ ID NO: 4 - DNA, 33 bp, synthetic construct
  source 1..33: mol_type = other DNA
tcgtcggcagcgtcagatgtgtataagagacag

SEQ ID NO: 5 - DNA, 19 bp, synthetic construct
  source 1..19: mol_type = other DNA
ctgtctcttatacacatct

SEQ ID NO: 6 - DNA, 33 bp, synthetic construct
  source 1..33: mol_type = other DNA
gacagagaatatgtgtagactgcgacggctgct

SEQ ID NO: 7 - DNA, 19 bp, synthetic construct
  source 1..19: mol_type = other DNA
tctacacatattctctgtc

SEQ ID NO: 8 - DNA, 67 bp, synthetic construct
  source 1..67: mol_type = other DNA
gtctcgtgggctcggagatgtgtataagagacagtttttttttttttttttttttttttttttttnn

SEQ ID NO: 9 - DNA, 25 bp, synthetic construct
  source 1..25: mol_type = other DNA
aaaaaaaaaaaaaaaaaaaaaaaaa

SEQ ID NO: 10 - DNA, 33 bp, synthetic construct; identical to SEQ ID NO: 4
  source 1..33: mol_type = other DNA

SEQ ID NO: 11 - DNA, 19 bp, synthetic construct
  source 1..19: mol_type = other DNA
ctgtctcttatacacatct

SEQ ID NO: 12 - DNA, 51 bp, synthetic construct
  source 1..51: mol_type = other DNA
aatgatacggcgaccaccgagatctacacnnnnnnnntcgtcggcagcgtc

SEQ ID NO: 13 - DNA, 47 bp, synthetic construct
  source 1..47: mol_type = other DNA
ggctcgggtgctctgnnnnnnnntagagcatacggcagaagacgaac

SEQ ID NO: 14 - DNA, 70 bp, synthetic construct
  source 1..70: mol_type = other DNA
aatgatacggcgaccaccgagatctacacnnnnnnnntcgtcggcagcgtcagatgtgtataagagacag

SEQ ID NO: 15 - DNA, 70 bp, synthetic construct
  source 1..70: mol_type = other DNA
ctgtctcttatacacatctgacgctgccgacgannnnnnnngtgtagatctcggtggtcgccgtatcatt

SEQ ID NO: 16 - DNA, 94 bp, synthetic construct
  source 1..94: mol_type = other DNA
aaaaaaaaaaaaaaaaaaaaaaaaaaaactgtctcttatacacatctccgagcccacgagacnnnnnnnnatctcgtatgccgtcttctgcttg

SEQ ID NO: 17 - DNA, 98 bp, synthetic construct
  source 1..98: mol_type = other DNA
caagcagaagacggcatacgagatnnnnnnnngtctcgtgggctcggagatgtgtataagagacagttttttttttttttttttttttttttttttnn

SEQ ID NO: 18 - DNA, 2382 bp, Drosophila melanogaster
  source 1..2382: mol_type = other DNA
tcaattctattcaaacaagtaaagtgaacacatcgctaagcgaaagctaagcaaacaaacaagcgcagctgaacaagctaaacaatctgcaataaagtgcaagttaaagtgaatcaattaaaagtaaccaacaaccaagtaattaaactaaaaactgcaactactgaaatcaaccaagaagtaattattgaagacaagaagagaactctgaatactttcaacaagtcgttaccgaggaagaagaactcacacacaatgcctgctattggaatcgatctgggcaccacctactcctgcgtgggtgtctaccaacatggcaaggtggagattatcgccaacgaccagggcaaccgcaccacgccgtcctacgtggctttcacagattcggaacgcctcatcggcgatccggctaagaaccaggtggccatgaaccccagaaacacagtgtttgacgccaagcgactgatcggccgaaaatacgacgaccccaagatcgcagaggacatgaagcactggcctttcaaggttgtaagcgacggcggaaagcccaagatcggggtggagtataagggtgagtccaagagatttgcccccgaggagatcagctcgatggtactgaccaagatgaaggagacggcggaggcatatctgggcgagagcatcacagacgcagtcatcacagttccagcctacttcaacgactcccagcgccaggctaccaaagacgccggtcacatcgccggcctgaatgtgctccgcatcatcaatgagcccacggcggcagcactggcctacggactggacaagaacctcaagggtgagcgcaatgtgcttatcttcgacttgggcggcggcaccttcgatgtctccatcctgaccatcgacgagggatcactgttcgaggtgcgctccaccgccggagacacacacttgggcggcgaggactttgacaaccggctagtcactcatctggcggacgagttcaagcgcaagtacaagaaggatctgcgctccaaccctcgcgccctacgacgcctcagaacagcagctgaacgggccaagcgcacactctcctccagcacggaggccaccatcgagattgacgcactgtttgagggccaagacttctacaccaaagtgagccgcgccaggtttgaggagctgtgcgcggacctcttccgcaacaccctgcagcctgtggagaaggccctcaacgatgccaagatggataagggtcagatccacgacatcgtgctcgtcggcggatccactcgcattcccaaggtgcaaagtctgctgcaggacttcttccacggcaagaacctcaacctatccatcaacccagacgaggcagttgcatacggagctgctgtgcaggccgctatcctcagcggagaccagagcggcaagatccaggacgtgctgctggtggacgtggccccactttcattgggaattgagaccgctggaggtgtaatgaccaagctgatcgagcgcaactgccgcattccgtgcaagcagactaagacgttctccacatacgcggacaaccagcccggagtctccattcaggtgtatgagggcgaacgtgcgatgacgaaggacaacaatgcattgggcaccttcgatctgtccggcattccacctgcaccaaggggtgtgccccagatagaagttaccttcgacttggacgccaatggaatcctgaacgtcagcgccaaggagatgagcacgggcaaggccaagaacatcacgatcaagaacgacaagggacggctctcgcaggccgagattgatcgcatggtgaacgaggctgaaaagtacgccgacgaggacgagaagcatcgccagcgaataacctctagaaatgccctggagagctacgtcttcaatgtgaagcaggccgtggaacaggcacctgctggcaaattggacgaggctgacaagaactccgtcttggacaagtgcaacgacactatccggtggctggacagcaacaccactgccgagaaggaggagttcgaccacaagctggaggagctcacccgccactgctcccccatcatgaccaagatgcatcagcagggtgcgggagctggagctggtggtccgggagcaaactgcggccagcaggcgggaggatttggaggctactctggacccacggtcgaggaggtcgactaaggccaaagagtctaatttttgttcatcaatgggttataacatatgggttatattataagtttgttttaagtttttgagactgataagaatgtttcgatcgaatattccatagaacaacaatagtattacctaattaccaagtcttaatttagcaaaaatgttattgcttatagaaaaaataaattatttattgaattt

SEQ ID NO: 19 - RNA, 45 nt, synthetic construct
  source 1..45: mol_type = other RNA
taatacgactcactatagggagaccacgatggcgatgttaatggg

SEQ ID NO: 20 - RNA, 43 nt, synthetic construct
  source 1..43: mol_type = other RNA
taatacgactcactatagggagaccacaggatcatgtgatctc

SEQ ID NO: 21 - RNA, 44 nt, synthetic construct
  source 1..44: mol_type = other RNA
ttaatacgactcactatagggagacagcctggagatggtgaact

SEQ ID NO: 22 - RNA, 44 nt, synthetic construct
  source 1..44: mol_type = other RNA
ttaatacgactcactatagggagatcaggcactcgacttttgtg

SEQ ID NO: 23 - RNA, 44 nt, synthetic construct
  source 1..44: mol_type = other RNA
ttaatacgactcactatagggagagcactggtcattggctaaac

SEQ ID NO: 24 - RNA, 45 nt, synthetic construct
  source 1..45: mol_type = other RNA
ttaatacgactcactatagggagacatctggataacgcttctgga

SEQ ID NO: 25 - DNA, 20 bp, Drosophila melanogaster
  source 1..20: mol_type = other DNA
agcatacaggcccaagatcg

SEQ ID NO: 26 - DNA, 20 bp, Drosophila melanogaster
  source 1..20: mol_type = other DNA
tgttgtcgatacccttgggc

SEQ ID NO: 27 - DNA, 20 bp, Drosophila melanogaster
  source 1..20: mol_type = other DNA
aaggaactcgttgaggctga

SEQ ID NO: 28 - DNA, 20 bp, Drosophila melanogaster
  source 1..20: mol_type = other DNA
cacctgtgtggagacaatgg

(19) United States
(12) Patent Application Publication
(10) Pub. No.: US 2025/0263782 A1
(43) Pub. Date: Aug. 21, 2025

(54) PROFILING RNA AT CHROMATIN TARGETS IN SITU BY ANTIBODY-TARGETED TAGMENTATION

(71) Applicant: Fred Hutchinson Cancer Center, Seattle, WA (US)

(72) Inventors: Steven Henikoff, Seattle, WA (US); Kami Ahmad, Seattle, WA (US); Nadiya Khyzha, Seattle, WA (US)

(21) Appl. No.: 18/857,328

(22) PCT Filed: Apr. 25, 2023
(86) PCT No.: PCT/US2023/066213

§ 371 Date: Oct. 16, 2024

Related U.S. Application Data
(60) Provisional application No. 63/334,582, filed on Apr. 25, 2022.

(51) Int. Cl. C12Q 1/6841; C12N 15/10

(52) U.S. Cl. CPC C12Q 1/6841; C12N 15/1065

(57) ABSTRACT

Embodiments of the present disclosure provide methods and kits for mapping a cellular RNA using a tethered enzyme complex. Embodiments of the method comprise binding a first recognition agent to an epitope of RNA or its associated proteins; binding a second recognition agent that specifically binds to the first recognition agent; and tethering an enzyme complex to the first recognition agent and/or the second recognition agent, wherein the enzyme complex enables reverse transcription to convert a mature transcript near the binding site of the first recognition agent to an RNA/DNA hybrid and tagmentation of the RNA/DNA hybrid for use in preparing sequencing libraries of the RNA/DNA hybrid.

### CROSS-REFERENCE(S) TO RELATED APPLICATION(S)

This application claims the benefit of U.S. Provisional Application Nos. 63/334,582, filed Apr. 25, 2022, the disclosure of which is hereby expressly incorporated herein by reference in its entirety.

### STATEMENT REGARDING SEQUENCE LISTING

The sequence listing associated with this application is provided in XML format in lieu of a paper copy and is hereby incorporated by reference into the specification. The name of the XML file containing the sequence listing is 1896-P70WO_Seq_List_20230425.xml. The XML file is 26 KB; was created on Apr. 25, 2023; and is being submitted via Patent Center with the filing of the specification.

### BACKGROUND

RNA levels are tightly regulated throughout their lifecycle to ensure proper gene expression. Factors influencing RNA post-transcriptionally include interaction with RNA binding proteins (RBPs), location within the nucleus, and post-transcriptional modifications. The most widely used strategy for assaying these factors is immunoprecipitation, whereby antibodies are used to pull down RNA associated with an epitope of interest from cell lysates. The recovered RNA is then purified and used for downstream applications such as Illumina sequencing. Variations of the immunoprecipitation protocol have been developed to study different types of RNA interactions. Examples include RNA immunoprecipitation (RIP) and UV cross-linking and immunoprecipitation (CLIP) for detecting RNA-protein interactions. Chromatin-specific immunoprecipitation assays include Profiling Interacting RNAs on Chromatin followed by deep sequencing (PIRCh-seq) and Chromatin RIP followed by high-throughput sequencing (ChRIP-seq) crosslink RNA to chromatin and assay RNA-chromatin interactions using antibodies targeting histone post-translational modifications. Immunoprecipitation assays for N⁶-methyladenosine (m6A) modified RNA include Methylated RNA Immunoprecipitation with next-generation sequencing (MeRIP-seq) and m6A-RIP-seq. Unfortunately, these immunoprecipitation-based methods require large sample inputs and optimization of crosslinking conditions.

There remains a need for sensitive in situ technologies that do not rely on cross-linking or immunoprecipitation to capture cellular RNAs. The present disclosure addresses these and related needs.

### SUMMARY

In accordance with the foregoing, in one aspect of the invention, the disclosure provides an in-situ method for mapping cellular RNA using a tethered enzyme complex. The method can comprise the steps of: binding a nucleus, an organelle, a cell, or a tissue to a solid support; permeabilizing the nucleus, the organelle, the cell or the tissue; binding a first recognition agent that binds an epitope of the RNA or its associated proteins; binding a second recognition agent that specifically binds to the first recognition agent, wherein the second recognition agent is conjugated to a biotin-binding moiety; tethering at least one molecule required for reverse transcription comprising a biotinylated oligonucleotide for cDNA synthesis priming and for PCR amplification to the second recognition agent; tethering a transposase fused to protein A (pA-transposase) comprising a first sequencing adapter sequence to the first recognition agent and the second recognition agent; allowing the at least one molecule required for reverse transcription to convert a mature transcript near the binding site of the first recognition agent to an RNA/DNA hybrid comprising a first sequencing adapter sequence and a priming sequence; allowing the pA-transposase to tagment the RNA/DNA hybrid; and preparing sequencing libraries of the RNA/DNA hybrid.

In some embodiments, the molecule required for reverse transcription can comprise a first sequencing adapter sequence, a priming sequence, and a biotinylated reverse transcriptase. In some embodiments, the reverse transcriptase can add three non-templated deoxycytidines (+CCC) to the 3′ end of a cDNA strand, which is then hybridized with an oligonucleotide comprising GGG nucleotides and a sequencing adapter for template-switching extension by the reverse transcriptase. In some embodiments, both the biotinylated priming sequence, comprising a second sequencing adapter, and the biotinylated reverse transcriptase can be tethered to a streptavidin-conjugated second recognition agent by a biotin-streptavidin interaction. In some embodiments, the molecule required for reverse transcription can comprise a biotinylated priming sequence comprising a second sequencing adapter. In some embodiments, the biotinylated priming sequence can be tethered to the streptavidin conjugated second recognition agent by a biotin-streptavidin interaction. In some embodiments, the pA-transposase can be tethered to the first recognition agent and the second recognition agent by Protein A in the pA-transposase binding to the first recognition agent and by Protein A in the pA-transposase binding to the second recognition agent.

In another aspect of the invention, the disclosure provides a kit. In some embodiments, the kit can comprise a first recognition agent; a streptavidin-conjugated second recognition agent; a transposase fused to protein A (pA-transposase) comprising a first sequencing adapter; a biotinylated priming sequence comprising a second sequencing adapter; a reverse transcriptase; an oligo(dT), a random priming oligonucleotide, and all required reagents for template switching, each packaged in a separate container; a solid support; and instructions directing the method as recited above.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A and 1B. Reverse Transcribe and Tagment (RT&Tag) general workflow. FIG. 1A. Schematic outlining the steps of RT&Tag: 1) nuclei are isolated and bound to Concanavalin A paramagnetic beads; 2) primary antibody binds epitope of interest; 3) streptavidin-conjugated secondary antibody binds to the primary antibody; 4) biotinylated oligo(dT)-adapter-B fusion oligonucleotide binds to the streptavidin conjugated secondary antibody; 5) protein A-Tn5 loaded with adapter-A binds to the primary and secondary antibodies; 6) simultaneous reverse transcription and tagmentation are then performed to generate RNA-cDNA hybrids that contain the two complementary adaptors; 7) sequencing libraries are amplified using PCR. FIG. 1B. Illustration showing applications of RT&Tag described in this work, which include identifying RNA-protein interactions, RNA-chromatin interactions, and RNA post-transcriptional modifications. This is contrasted to immunoprecipitation-based techniques which require a separate method for targeting each type of interaction. Sequence identification: TTTTTT (SEQ ID NO: 1), AAAAAAAAA (SEQ ID NO: 2), TTTTTTTTT (SEQ ID NO: 3).

FIGS. 2A through 2I. Reverse Transcribe and Tagment (RT&Tag) captures the interaction between MSL2 and the roX2 noncoding RNA. FIG. 2A. Illustration showing RT&Tag being used to capture the interaction between MSL2 and roX2. FIG. 2B. Tapestation gel image and corresponding electropherogram showing size distribution of the MSL2 RT&Tag libraries after two rounds of 0.8× bead clean-up. In the absence of reverse transcriptase (RT), no libraries are produced. FIG. 2C. Pie chart showing the proportion of MSL2 RT&Tag reads (n=4) aligning to regions classified as either exonic, intronic, or intergenic. FIG. 2D. Density plot showing the distribution of aligned MSL2 RT&Tag reads (n=4) scaled over Drosophila gene bodies. A clear bias towards the 3′ end of genes is observed. A small bump around 15-20^{th} percentile may be explained by internal priming at A-rich stretches, especially those found with the 18s and 26s rRNAs. FIG. 2E. Principal component analysis showing separation between IgG and MSL2 RT&Tag samples along the first principal component and separation between replicates in the second principal component. The first two and last two replicates have been sequenced on two separate flow cells and hence a batch effect may be observed. FIG. 2F. Volcano plot showing transcripts differentially enriched for MSL2 over IgG RT&Tag (fold change >2, FDR <0.05, n=4). Transcripts enriched for MSL2 are within the dashed lined box, nonenriched are outside the dashed lined box, and depleted are non-filled circles. FIG. 2G. Genome browser track showing the distribution of MSL2 and IgG RT&Tag signal over the gene body of roX2. Combined reads from 4 replicates are shown. FIG. 2H. Karyoplots showing the bins (50 bp) where MSL2 RT&Tag signal is 4-fold over IgG plotted over the Drosophila chromosomes. FIG. 2I. Profile plots showing the MSL2 (top) and H4K16ac (bottom) CUT&Tag signal around the TSS (top) and gene bodies (bottom) of MSL2 RT&Tag enriched or nonenriched transcripts.

FIGS. 3A through 3H. Reverse Transcribe and Tagment (RT&Tag) captures transcripts within polycomb domains. FIG. 3A. Illustration showing RT&Tag being used to capture transcripts within H3K27me3 demarcated Polycomb domains. FIG. 3B. Volcano plot showing transcripts that are differentially enriched for H3K27me3 RT&Tag over IgG (fold change >2, FDR <0.05, n=5). Genes enriched for H3K27me3 are highlighted in the dashed lined box, nonenriched are outside the dashed lined box, and depleted are non-filled circles. The two most highly significant transcripts are labelled. FIG. 3C. Genome browser track showing the distribution of H3K27me3 and IgG RT&Tag signal over the gene bodies of CR43334 and CR42862. Combined reads from 5 replicates are shown. FIG. 3D. Bar graph showing the number of H3K27me3 enriched transcripts that are protein coding or noncoding. FIG. 3E. Boxplot showing the RNA-seq expression levels (Counts per million, CPM) of H3K27me3 enriched or nonenriched transcripts. *p<0.05, unpaired t-test, n=5. FIG. 3F. Profile plots showing the H3K27me3 (left), H3K36me3 (middle) and H3K4me3 (right) CUT&Tag signal around the gene bodies or TSS of genes that were categorized as being enriched for H3K27me3 RT&Tag or nonenriched. FIG. 3G. Boxplots showing the IgG and H3K27me3 RT&Tag signal (Counts per million, CPM) for the HOX cluster genes. *FDR<0.05, n=5. FIG. 3H. Profile showing the H3K27me3 (left), H3K36me3 (center) and H3K4me3 (right) signal over the gene bodies or TSS of H3K27me3 RT&Tag enriched transcripts that have high or low levels of H3K27me3 CUT&Tag signal over their gene bodies.

FIGS. 4A through 4E. Reverse Transcribe and Tagment (RT&Tag) captures transcripts enriched for the m6A posttranscriptional modification. FIG. 4A. Illustration showing RT&Tag being used to capture transcripts enriched for the m6A post-transcriptional modification. FIG. 4B. Volcano plot showing genes that are differentially enriched for m6A over IgG RT&Tag (fold change >1.5, FDR <0.05, n=3). Genes enriched for m6A are located to the right of the vertical bold line and above the horizontal bold line, nonenriched are between the vertical bold lines and below the horizontal bold line (i.e., both between and outside of the vertical bold lines), and depleted are to the left of the bold line and above the horizontal bold line. Genes previously shown to be enriched or depleted for m6A are labelled. FIG. 4C. Genome browser track showing the distribution of m6A and IgG RT&Tag reads over the gene body of aqz and Syx1A. Combined reads from 3 replicates are shown. FIG. 4D. Dot plot showing the top 5 GO biological process (top) and molecular function (bottom) terms associated with m6A enriched and m6A depleted transcripts. The dot size corresponds to the gene ratio (#genes related to GO term/total number of m6A enriched or depleted genes). FIG. 4E. Profile plots showing the METTL3 CUT&Tag signal at the TSS of genes that are enriched, nonenriched, or depleted for m6A.

FIGS. 5A through 5H. Genes of methylated transcripts are characterized by promoter proximally paused RNA Polymerase II. FIG. 5A. Profile plots showing METTL3 (left) and total RNA polymerase II (RNAPolII, right) CUT&Tag signal at the TSS of the top 25% expressed genes. FIG. 5B. Violin plots showing the RNA-seq expression levels (counts per million, CPM) of genes that are depleted, enriched, or nonenriched for m6A. *p<0.05, unpaired t-test. FIG. 5C. Profile plots showing the H3K36me3 (top) and H3K4me3 (bottom) CUT&Tag signal over the gene bodies or at the TSS of genes that are enriched, non-enriched, or depleted for m6A. FIG. 5D. Genome browser tracks showing the IgG, METTL3, and RNAPolII CUT&Tag signal over the gene bodies of Hsp70 genes with no heat shock (no HS) or after 15 minutes of heat shock (HS). FIG. 5E. Bar graph showing the IgG and m6A RT&Tag signal for Hsp70 with no heat shock (no HS) and after 15 minutes of heat shock (HS). FIG. 5F. Profile plot showing GAGA factor (GAF) CUT&Tag signal at the TSS of m6A enriched, nonenriched, and depleted transcripts. FIG. 5G. Profile plot showing RNAPolII CUT&Tag signal over the gene bodies of m6A enriched, nonenriched, and depleted transcripts. FIG. 5H. Schematic showing how the promoter proximal pausing index (PI) was calculated (left). PI was calculated by dividing the promoter (+/−250 bp around the TSS) RNAPolII CUT&Tag signal over the gene body RNAPolII CUT&Tag signal. Violin plots displaying the PI of m6A enriched, nonenriched, and depleted transcripts (right). *p<0.05, unpaired t-test.

FIGS. 6A through 6C. Optimization of Reverse Transcribe and Tagment (RT&Tag). FIG. 6A. Performance comparison of RT&Tag using biotinylated or un-biotinylated oligo(dT)-adaptor B fusion oligonucleotides based on the following metrics: roX2 enrichment for MSL2 (left) and number of differentially enriched transcripts for K27me3 (right). Both experiments were performed using reverse transcription performed at the same time as tagmentation (CoTagRT) approach. FIG. 6B. Performance comparison of RT&Tag if reverse transcription is performed prior to addition of pA-Tn5 (preTagRT) or if reverse transcription is performed at the same time as tagmentation (CoTagRT). Both experiments were performed using un-biotinylated oligo(dT)-adaptor B fusion oligonucleotides. Performance of RT&Tag was assessed based on the following metrics: roX2 enrichment for MSL2 (top left), number of differentially enriched transcripts for K27me3 (top right) and number of differentially enriched transcripts for m6A (bottom) with pre-TagRT versus Co-TagRT. Differential enrichment was defined as >2-fold change for K27me3 or >1.5-fold change for m6A, <0.05 FDR. FIG. 6C. Density plots showing the distribution of aligned MSL2 (top) and H3K27me3 (bottom) RT&Tag reads (n=2) scaled over Drosophila gene bodies for biotinylated oligo(dT) CoTagRT (left), unbiotinylated oligo(dT) CoTagRT (center), and unbiotinylated oligo(dT) preTagRT (right) RT&Tag variations. A clear bias towards the 3′ end of genes is observed under all conditions.

FIG. 7. Construction of Reverse Transcribe and Tagment (RT&Tag) libraries. Schematic showing how RT&Tag libraries are generated. During reverse transcription the oligo(dT)-ME-B fusion oligonucleotide binds to the poly(A) tail of RNA. Anchored oligo(dT) is used to ensure binding at the start of the poly(A) tail. Through the process of reverse transcription, the ME-B sequence gets appended to the cDNA. The RNA/cDNA hybrid then gets tagmented with ME-A loaded Tn5. Sequencing libraries are then amplified using primers complementary to the i5 and i7 sequences. The libraries are sequenced using 50 base pair single-end sequencing with the read originating from the i5 side. Figure sequences are provided in Table

FIGS. 8A and 8B. H3K27me3 and m6A Reverse Transcribe and Tagment (RT&Tag) signal. FIG. 8A. Pie chart showing the proportion of H3K27me3 (left, n=5) and m6A (right, n=3) RT&Tag reads aligning to regions classified as either exonic, intronic, or intergenic. FIG. 8B. Density plots showing the distribution of aligned H3K27me3 (left, n=5) and m6A (right, n=3) RT&Tag reads scaled over Drosophila gene bodies.

FIGS. 9A and 9B. Reverse Transcribe and Tagment (RT&Tag) captures the interaction between MSL2 and transcripts within its vicinity. FIG. 9A. Boxplot showing the genomic distance from the gene body of MSL2 enriched or nonenriched transcripts to the nearest MSL2 peak. *p<0.05. FIG. 9B. Genome browser tracks showing the distribution of IgG and MSL2 RT&Tag signal as well as MSL2 and H4K16ac CUT&Tag signal over the ph-d and pcx gene bodies.

FIGS. 10A through 10D. Performance comparison of MSL2 Reverse Transcribe and Tagment (RT&Tag) to RIP-seq. FIG. 10A. Volcano plot showing transcripts differentially enriched for MLE RIP-seq over input (fold change >2, FDR <0.05, n=3, GSE143455). Transcripts enriched for MLE are to the right of the vertical bold line and above the horizontal bold line, nonenriched are between the vertical bold lines and below the horizontal bold line (i.e., both between and outside of the vertical bold lines), and depleted are to the left of the vertical bold line and above the horizontal bold line. FIG. 10B. Table comparing MSL2 RT&Tag and MLE RIP-seq in terms of number of cells, number of reads, and roX2 fold change enrichment for MSL2/MHLE over control. FIG. 10C. Venn diagram showing the overlap between transcripts enriched for MSL2 RT&Tag and MLE RIP-seq with roX1 and roX2 being enriched in both. FIG. 10D. Pie charts showing the chromosomal distribution of transcripts uniquely enriched for MSL2 RT&Tag (left) and MLE RIP-seq (right).

FIGS. 11A and 11B. H3K27me3 Reverse Transcribe and Tagment (RT&Tag) performance with decreasing number of nuclei input. FIG. 11A. Genome browser tracks showing the distribution of IgG and H3K27me3 RT&Tag signal from 100,000, 25,000, or 5000 nuclei over the gene bodies of CR43334 and CR42862. Combined reads from 2 replicates are shown. FIG. 11B. Boxplots showing the IgG and H3K27me3 RT&Tag signal (Counts per million, CPM) from 100,000, 25,000, or 5000 nuclei for the HOX cluster genes. *FDR<0.05, n=2.

FIGS. 12A through 12C. Reverse Transcribe and Tagment (RT&Tag) captures transcripts within polycomb domains. FIG. 12A. Dot plot showing the top 10 GO biological process terms associated with H3K27me3-enriched transcripts. The dot size corresponds to the gene count. FIG. 12B. Profile plot showing the H3K27me3 CUT&Tag signal over the gene bodies of the top 25% expressed genes. FIG. 12C. Boxplot showing the RNA-seq expression levels (Counts per million, CPM) of H3K27me3-RT&Tag enriched transcripts that had either high (>9 read counts) or low (<9 read counts) H3K27me3 CUT&Tag signal over their gene bodies. *p<0.05, unpaired t-test.

FIG. 13. M6A Reverse Transcribe and Tagment (RT&Tag) performance with decreasing number of nuclei input. Genome browser tracks showing the distribution of IgG and m6A RT&Tag signal from 100,000, 25,000, or 5000 nuclei over the gene bodies of aqz and Syx1A. Combined reads from 2 replicates are shown.

FIGS. 14A through 14F. Genes of methylated transcripts are characterized by promoter proximally paused RNA Polymerase II. FIG. 14A. Bar plot showing Mettl3 expression measured by real time PCR in control RNAi and Mettl3 RNAi S2 cells. Data is plotted relative to control RNAi. FIG. 14B. Profile plot showing IgG and METTL3 CUT&Tag signal over the gene bodies of m6A depleted genes. FIG. 14C. Pearson correlation between RNAPolII and METTL3 CUT&Tag signal at the promoters of top 25% expressed genes. FIG. 14D. Sequence of Hsp70Aa with RRACH motifs highlighted in grey (SEQ ID NO: 18). FIG. 14E. MEME motif logos found to be enriched within the promoters of m6A enriched transcripts relative to those of m6A depleted transcripts using the differential enrichment mode setting. FIG. 14F. Violin plots displaying the promoter proximal pausing index (PI) m6A-enriched transcripts broken down into quartiles based on their RNA-seq expression levels. PI was calculated by dividing the promoter (+/−250 bp around the TSS) RNAPolII CUT&Tag signal over the gene body RNAPolII CUT&Tag signal.

FIGS. 15A and 15B. Reverse Transcribe and Tagment (RT&Tag) detects mammalian IncRNAs. H3K27me3-tethered RT&Tag was performed with female K562 cells. The MST and TSIX IncRNAs are enriched. FIG. 15A. XLST/TSIX are highly enriched in Polycomb domains. FIG. 15B. RT&Tag detects very long transcripts in Polycomb domains. Specifically, XLST/TSIX are uniquely bound to Polycomb domains.

FIGS. 16A and 16B. New transcripts from silenced domains have short half-lives. (SH)-Linked Alkylation for the Metabolic sequencing of RNA-Reverse Transcribe and Tagment (SLAM-RT&Tag) was performed on K562 cells. FIG. 16A. The cumulative distribution plot shows relative change in labeling ratios (an estimate of half-life) for H3K27me3-tethered SLAM-RT&Tag or IgG controls. FIG. 16B. SLAM-RT&Tag (Thiol(sh)-linked alkylation for the metabolic sequencing of RNA (SLAM-seq) Based on Herzog et al. (Herzog, V., Reichholf, B., Neumann, T. et al. Thiol-linked alkylation of RNA to assess expression dynamics. Nat Methods 14, 1198-1204 (2017)).

FIGS. 17A and 17B. An expanded RT&Tag toolkit. FIG. 17A. Design of RT&Tag with SMART 5′ adapter and random hexamer priming. mRNA (i.e., poly-A tail) is reverse transcribed from an oligo-dT-A adapter (1) or from random hexamers (rh, 2) and extended across a SMART-A adapter. Tagmentation from chromatin-tethered pA-Tn5 completes library molecules. FIG. 17B. Landscape of H3K27me3-tethered RT&Tag read distribution and IgG controls across the XIST gene for oligo-dT-primed RT&Tag (i.e., top IgG and H3K27me3) and for 5′RT&Tag (i.e., bottom IgG and H3K27me3).

### DETAILED DESCRIPTION

Whereas techniques to map chromatin-bound proteins are well-developed, mapping chromatin-associated RNAs remains a challenge. This disclosure is based on the inventors development of a platform technique referred to as “Reverse Transcribe & Tagment” (RT&Tag), in which RNAs associated with a chromatin epitope are targeted by an antibody followed by a protein A-Tn5 transposome. Localized reverse transcription generates RNA/cDNA hybrids that are subsequently tagmented for sequencing by Tn5. As described in more detail below, the inventors demonstrate the utility of RT&Tag in Drosophila cells for capturing the noncoding RNA roX2 with the dosage compensation complex and maturing transcripts associated with silencing histone modifications. It is also demonstrated that RT&Tag can detect N6-methyladenosine (m6A)-modified mRNAs, and show that genes producing methylated transcripts are characterized by extensive promoter pausing of RNA polymerase II. The high efficiency of in situ antibody tethering and tagmentation makes RT&Tag especially suitable for rapid low-cost profiling of chromatin-associated RNAs from small samples.

### Methods for Mapping Cellular RNA

In accordance with the foregoing, in one aspect of the invention, the disclosure provides an in-situ method for mapping cellular RNA and its associated proteins using a tethered enzyme complex. The method can comprise the steps of: binding a nucleus, an organelle, a cell, or a tissue to a solid support; permeabilizing the nucleus, the organelle, the cell or the tissue; binding a first recognition agent that binds an epitope of the RNA or its associated proteins; binding a second recognition agent that specifically binds to the first recognition agent, wherein the second recognition agent is conjugated to a biotin-binding moiety; tethering at least one molecule required for reverse transcription comprising a biotinylated oligonucleotide for cDNA synthesis priming and for PCR amplification to the second recognition agent; tethering a transposase fused to protein A (pA-transposase) comprising a first sequencing adapter sequence to the first recognition agent and the second recognition agent; allowing the at least one molecule required for reverse transcription to convert a mature transcript near the binding site of the first recognition agent to an RNA/DNA hybrid comprising a first sequencing adapter sequence and a priming sequence; allowing the pA-transposase to tagment the RNA/DNA hybrid; and preparing sequencing libraries of the RNA/DNA hybrid.

As used herein, cellular RNA or its associated proteins refers to chromatin-associated RNA, free RNA, including cytoplasmic RNA, chromatin modifications, or RNA-binding proteins. Chromatin-associated RNA refers to RNA that is bound directly or indirectly to chromatin. Chromatin-associated RNA can be bound directly to the chromatin, for example by base-pairing interactions with DNA of the chromatin (either single-stranded or double-stranded DNA of the chromatin), or by RNA-protein interactions with protein of the chromatin. Alternatively, chromatin-associated RNA can be bound indirectly to the chromatin, for example as part of a complex with a protein which is itself bound directly or indirectly to the chromatin, or as part of a network of nucleic acids that are bound to the chromatin. In some embodiments, the chromatin-associated RNA comprises a non-protein-coding RNA (ncRNA). Examples of ncRNAs can include, but are not limited to, long noncoding RNAs (IncRNAs), chromatin-enriched RNAs (cheRNAs), small noncoding RNAs (small ncRNAs), micro RNAs (miRNAs), small interfering RNAs (siRNAs), PIWI-interacting RNAs, ribosomal RNAs (rRNAs), transfer RNAs (tRNAs), small nuclear RNAs (snRNAs), small nucleolar RNAs (snoRNAs), ribozymes.

Chromatin modifications, as understood by one of ordinary skill in the art disrupt chromatin contacts or affect the recruitment of nonhistone proteins to chromatin. In some embodiments, chromatin modifications include but are not limited to acetylation, methylation (lysines), methylation (arginines), phosphorylation, ubiquitylation, sumoylation, ADP ribosylation, deamination, or proline isomerization.

RNA-binding proteins can include proteins that bind to double or single stranded RNA in cells and participate in forming ribonucleoprotein complexes as understood by one of ordinary skill in the art.

Free RNA is any RNA that is not associated with chromatin-directly or indirectly—as understood by one of ordinary skill in the art.

In some embodiments, the nucleus, the organelle, the cell or the tissue is obtained from a eukaryotic sample. In other embodiments, the nucleus, the organelle, the cell or the tissue is obtained from a human sample. In still other embodiments, the nucleus, the organelle, the cell or the tissue is obtained from a non-diseased tissue or sample. In some embodiments, the nucleus, organelle, cell, or tissue is or is from a peripheral tissue or cell, e.g., a peripheral blood mononuclear cell. In some embodiments, the nucleus, organelle, cell, or tissue is or is from cultured cells, e.g., primary cells.

In still other embodiments, the nucleus, the organelle, the cell or the tissue is obtained from a biological sample. In some embodiments, the biological sample can comprise body fluid, including but not limited to blood, serum, plasma, urine, saliva, semen, prostatic fluid, nipple aspirate fluid, lachrymal fluid, perspiration, feces, cheek swabs, cerebrospinal fluid, cell lysate samples, amniotic fluid, gastrointestinal fluid, biopsy tissue, lymphatic fluid, or cerebrospinal fluid. In still other embodiments, the biological sample can be any sample from which cellular RNA can be isolated. In some embodiments, the biological sample is isolated from a subject with a disease or disorder associated with changes in one or more cellular RNAs. In other embodiments, the biological sample is isolated from a subject's tissue or organ affected by a disease or disorder associated with changes in one or more cellular RNAs. The biological sample can be obtained from the diseased organ or tissue by any means known in the art, including but not limited to biopsy, aspiration, and surgery.

In other embodiments, the biological sample is not from a tissue or organ affected by a disease or disorder associated with changes in one or more cellular RNAs. In some embodiments, the biological sample (e.g., cells) can serve as a proxy for the diseased biological sample (e.g., diseased cells). In other embodiments, the biological sample (e.g., cells) can be more readily accessible than the diseased biological sample (e.g., diseased cells). For example, the biological sample (e.g., cells) can be obtained without the needs for complicated or painful procedures such as biopsies, such samples can include but are not limited to peripheral blood mononuclear cells.

In still other embodiments, the nucleus, the organelle, the cell or the tissue is obtained from a subject of interest. In some embodiments, the subject of interest can be any subject for which the methods of the present invention are desired. In some embodiments, the subject of interest is a mammal, e.g., a human. In some embodiments, the subject of interest is a laboratory animal, e.g., a mouse, rat, dog, or monkey, e.g., an animal model of a disease. In certain embodiments, the subject of interest can be one that has been diagnosed with or is suspected of having a disease or disorder. In some embodiments, the subject of interest can be one that is at risk for developing a disease or disorder, e.g., due to genetics, family history, exposure to toxins, etc.

In some embodiments, the first recognition agent can be, but is not limited to, an antibody, an aptamer, or a nanobody that can specifically bind to an epitope of the cellular RNA or its associated proteins. As understood by one of ordinary skill in the art, the disclosed method will work with any first recognition agent.

In some embodiments, the second recognition agent can be, but is not limited to, an antibody, an aptamer, or a nanobody that can specifically bind to the first recognition agent. As understood by one of ordinary skill in the art, the disclosed method will work with any second recognition agent. In some embodiments, the second recognition agent can be conjugated to a biotin-binding moiety. A biotin-biding moiety is any moiety, as understood by one of ordinary skill in the art, capable of binding to biotin. In still other embodiments, the biotin-binding moiety can be conjugated to the second recognition agent in any manner that will allow for specific binding of the corresponding biotin-conjugated agent. One of ordinary skill in the art would be able to determine how the biotin-binding moiety is conjugated to the second recognition agent.

As used herein, tethering refers to attaching at least one molecule required for reverse transcription to a second recognition agent, wherein the tethered molecule at least (1) anneals to the RNA for synthesis of cDNA and (2) converts the RNA to cDNA. In some embodiments, the molecule comprises at least a biotinylated priming sequence. In some embodiments, the biotinylated priming sequence can be any biotinylated oligonucleotide for cDNA synthesis. In still other embodiments, the biotinylated priming sequence can be a biotinylated random hexamer. In still other embodiments, the biotinylated priming sequence can a biotinylated olido(dT). In still other embodiments, the biotinylated priming sequence further comprises a sequencing adapter sequence for PCR amplification. One of ordinary skill in the art would be able to identify adapter sequences for use in the oligonucleotide for PCR amplification. One of ordinary skill in the art would be able to determine how biotin is best conjugated to the oligonucleotide to allow for cDNA synthesis priming.

In some embodiments, the molecule required for reverse transcription can comprise a reverse transcriptase, wherein the reverse transcriptase converts a mature transcript near the binding site of the first recognition agent to an RNA/DNA hybrid. One of ordinary skill in the art would be able to determine how biotin is best conjugated to enable converting a mature transcript to an RNA/DNA hybrid.

In some embodiments, a biotinylated priming sequence and a biotinylated reverse transcriptase are tethered to the second recognition agent. In some embodiments, a biotinylated priming sequence is tethered to the second recognition agent and an un-tethered reverse transcriptase can be added to convert a mature transcript to an RNA/DNA hybrid.

In some embodiments, tethering refers to attaching a transposase fused to protein A (pA-transposase) to the first recognition agent and the second recognition agent. In some embodiments, the transposase fused to protein A further comprises a sequencing adapter sequence for PCR amplification. One of ordinary skill in the art would be able to identify adapter sequences for use in the pA-transposase for PCR amplification.

In some embodiments, the molecule required for reverse transcription can comprise a first sequencing adapter sequence, a priming sequence, and a biotinylated reverse transcriptase. In some embodiments, the reverse transcriptase can add three non-templated deoxycytidines (+CCC) to the 3′ end of a cDNA strand, which is then hybridized with an oligonucleotide comprising GGG nucleotides and a sequencing adapter for template-switching extension by the reverse transcriptase.

In some embodiments, both the biotinylated priming sequence, comprising a second sequencing adapter, and the biotinylated reverse transcriptase can be tethered to a streptavidin-conjugated second recognition agent by a biotin-streptavidin interaction.

In some embodiments, the molecule required for reverse transcription can comprise a biotinylated priming sequence comprising a second sequencing adapter. In some embodiments, the biotinylated priming sequence can be tethered to the streptavidin conjugated second recognition agent by a biotin-streptavidin interaction. In some embodiments, the pA-transposase can be tethered to the first recognition agent and the second recognition agent by Protein A in the pA-transposase binding to the first recognition agent and by Protein A in the pA-transposase binding to the second recognition agent.

In another aspect of the invention, the disclosure provides a kit. In some embodiments, the kit can comprise a first recognition agent; a streptavidin-conjugated second recognition agent; a transposase fused to protein A (pA-transposase) comprising a first sequencing adapter; a biotinylated priming sequence comprising a second sequencing adapter; a reverse transcriptase; an oligo(dT), a random priming oligonucleotide, and all required reagents for template switching, each packaged in a separate container; a solid support; and instructions directing the method as recited above.

In some embodiments, reagents can be, for example, buffers, primers, enzymes, dNTPs, carrier RNA, and other active agents and organics that facilitate various steps of the disclosed reactions.

In some embodiments, instructions for use can be found in the kit. In other embodiments, the instructions for use can be found through an appropriate website.

### Additional Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented in the 5′ to 3′ direction, from left to right, unless specifically indicated otherwise.

Except as otherwise indicated, standard methods known to those skilled in the art can be used for production of recombinant and synthetic polypeptides, antibodies or antigen-binding fragments thereof, manipulation of nucleic acid sequences, production of transformed cells, the construction of nucleosomes, and transiently and stably transfected cells. Such techniques are known to those skilled in the art. See, e.g., Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) for definitions and terms of art. Additionally, definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710). In case of conflict, the terms in the specification will control.

All publications, patent applications, patents, nucleotide sequences, amino acid sequences and other references mentioned herein are incorporated by reference in their entirety.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element. Additionally, the words “herein,” “above,” and “below,” and words of similar import, when used in this application, shall refer to this application as a whole and not to any particular portions of the application. The word “about” indicates a number within range of minor variation above or below the stated reference number. For example, in some embodiments “about” can refer to a number within a range of 10%, 9%, 8%, 7%, 6%, 5%, 4%, 3%, 2%, or 1% above or below the indicated reference number.

To facilitate review of the various embodiments of this disclosure, the following explanations of specific terms are provided.

As used herein, the term “polypeptide” encompasses both peptides and proteins, unless indicated otherwise.

A “nucleic acid” or “nucleotide sequence” is a sequence of nucleotide bases, and can be RNA, DNA or DNA-RNA hybrid sequences (including both naturally occurring and non-naturally occurring nucleotide), but is preferably either single or double stranded DNA sequences.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

The term “specifically binds” refers to, with respect to an antigen, the preferential association of an affinity reagent, in whole or part, with a specific antigen, such as a specific a post-translational modification of RNA. A specific binding affinity agent binds substantially only to a defined target, such as a specific chromatin associated factor or marker. It is recognized that a minor degree of non-specific interaction can occur between a molecule, such as a specific affinity reagent, and a non-target antigen. Nevertheless, specific binding can be distinguished as mediated through specific recognition of the antigen. Specific binding typically results in greater than 2-fold, such as greater than 5-fold, greater than 10-fold, or greater than 100-fold increase in amount of bound affinity reagent (per unit time) to a target antigen, such as compared to a non-target antigen. A variety of immunoassay formats are appropriate for selecting affinity reagent specifically reactive with a particular antigen. For example, solid-phase ELISA immunoassays are routinely used to select antibodies specifically immunoreactive with a protein. See Harlow & Lane, Antibodies, A Laboratory Manual, Cold Spring Harbor Publications, New York (1988), for a description of immunoassay formats and conditions that can be used to determine specific reactivity.

An “antibody” is a polypeptide ligand that includes at least a light chain or heavy chain immunoglobulin variable region and specifically binds an epitope of an antigen, such as a chromatin associated marker or another affinity reagent. The term “antibody” encompasses antibodies, derived from any antibody-producing mammal (e.g., mouse, rat, rabbit, and primate including human), that specifically bind to an antigen of interest (e.g., a chromatin associated marker or another affinity reagent). Exemplary antibody types include multi-specific antibodies (e.g., bispecific antibodies), humanized antibodies, murine antibodies, chimeric, mouse-human, mouse-primate, primate-human monoclonal antibodies, and anti-idiotype antibodies.

DNA sequencing refers to the process of determining the nucleotide order of a given DNA molecule. Generally, the sequencing can be performed using automated Sanger sequencing (e.g., using AB 13730x1 genome analyzer), pyrosequencing on a solid support (e.g., using 454 sequencing, Roche), sequencing-by-synthesis with reversible terminations (e.g., using ILLUMINA® Genome Analyzer), sequencing-by-ligation (e.g., using ABI SOLiD®) or sequencing-by-synthesis with virtual terminators (e.g., using HELISCOPE®) other next generation sequencing techniques for use with the disclosed methods include, Massively parallel signature sequencing (MPSS), Polony sequencing, Ion Torrent semiconductor sequencing, DNA nanoball sequencing, Heliscope single molecule sequencing, Single molecule real time (SMRT) sequencing, and Nanopore DNA sequencing

The term “nucleic acid” (molecule or sequence) refers to a deoxyribonucleotide or ribonucleotide polymer including without limitation, cDNA, mRNA, genomic DNA, and synthetic (such as chemically synthesized) DNA or RNA or hybrids thereof. The nucleic acid can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Nucleotides include those nucleotides containing modified bases, modified sugar moieties, and modified phosphate backbones, for example as described in U.S. Pat. No. 5,866,336 to Nazarenko et al. Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxy acetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, 2,6-diaminopurine and biotinylated analogs, amongst others. Examples of modified sugar moieties which can be used to modify nucleotides at any position on its structure include, but are not limited to arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

The terms peptide/protein/polypeptide refer to a polymer of amino acids and/or amino acid analogs that are joined by peptide bonds or peptide bond mimetics. The twenty naturally occurring amino acids and their single-letter and three-letter designations known in the art.

Sequence identity and similarity between multiple nucleic acid or polypeptide sequences can be determined. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5: 151-3, 1989; Corpet et al, Nuc. Acids Res. 16: 10881-90, 1988; Huang et al. Computer Appls. in the Biosciences 8, 155-65, 1992; and Pearson et al, Meth. Mol. Bio. 24:307-31, 1994. Altschul et al, J. Mol. Biol. 215:403-10, 1990, presents a detailed consideration of sequence alignment methods and homology calculations.

The NCBI Basic Local Alignment Search Tool (BLAST) (Altschul et al, J. Mol. Biol. 215:403-10, 1990) is available from several sources, including the National Center for Biological Information (NCBI, National Library of Medicine, Building 38 A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554* 100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (i.e., 15±20*100=75)

The term “transposome” refers to a transposase-transposon complex. A conventional way for transposon mutagenesis usually places the transposase on the plasmid. In some such systems, the transposase can form a functional complex with a transposon recognition site that is capable of catalyzing a transposition reaction. The transposase or integrase can bind to the transposase recognition site and insert the transposase recognition site into a target nucleic acid in a process sometimes termed “tagmentation”.

The phrase “under conditions that permit binding” refers to any environment that permits the desired activity, for example, conditions under which two or more molecules, such as nucleic acid molecules and/or protein molecules, can bind. Such conditions can include specific concentrations of salts and/or other chemicals that facilitate the binding of molecules.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

All publications cited herein and the subject matter for which they are cited are hereby specifically incorporated by reference in their entireties.

### EXAMPLES

The following examples are provided to illustrate certain particular features and/or embodiments of the disclosure. The examples should not be construed to limit the disclosure to the particular features or embodiments described.

### Example 1

This Example describes the development of “Reverse Transcribe & Tagment” (RT&Tag), in which RNAs associated with a chromatin epitope are targeted by an antibody followed by a protein A-Tn5 transposome. Characterization of the performance of RT&Tag platform establishes that high efficiency of in situ antibody tethering and tagmentation makes this platform especially suitable for rapid low-cost profiling of chromatin-associated RNAs from small samples.

Cleavage Under Targets and Tagmentation (CUT&Tag) is an enzyme-tethering strategy developed to profile the binding sites of chromatin proteins within intact nuclei (see e.g., WO2019060907; Kaya-Okur, H. S. et al. CUT&Tag for efficient epigenomic profiling of small samples and single cells. Nat Commun 10, 1930 (2019), each of which is incorporated herein by reference in its entirety). CUT&Tag bypasses immunoprecipitation and instead uses antibodies to tether a protein A-Tn5 transposase fusion protein in situ. Tn5 undergoes a tagmentation reaction where genomic DNA is cleaved and tagged with sequencing adaptors. These sequencing adaptors are then used to generate Illumina sequencing libraries. However, Tn5 also contains an RNase H-like domain that can bind and tagment reverse transcribed RNA/cDNA hybrids (Di, L. et al. RNA sequencing by direct tagmentation of RNA/DNA hybrids. Proc. Natl. Acad. Sci. U.S.A. 117, 2886-2893 (2020); Lu, B. et al. Transposase-assisted tagmentation of RNA/DNA hybrid duplexes. Elife 9 (2020)).

The inventors developed Reverse Transcribe & Tagment (RT&Tag), a proximity labeling tool for capturing RNA interactions within intact nuclei. RT&Tag follows the framework of CUT&Tag but is adapted to capture signal from RNA instead of genomic DNA. Relative to RIP-based immunoprecipitation methods, RT&Tag requires few cells and a low number of sequencing reads, while capturing interactions within intact nuclei. In this work, the general utility of RT&Tag is demonstrated by applying it to a variety of RNA- and chromatin-dependent phenomena in Drosophila S2 nuclei. Specifically, RT&Tag was used to target the dosage compensation complex, Polycomb chromatin domains, and the m6A RNA post-transcriptional modification. Surprisingly, it was found that binding of the m6A writer, METTL3, is not sufficient for RNA methylation. Instead, it was revealed that RNA Polymerase II pausing is a strong predictor of m6A mark deposition. This finding illustrates the potential of RT&Tag to empower research in the fields of epigenetics and RNA biology.

### RT&Tag General Workflow

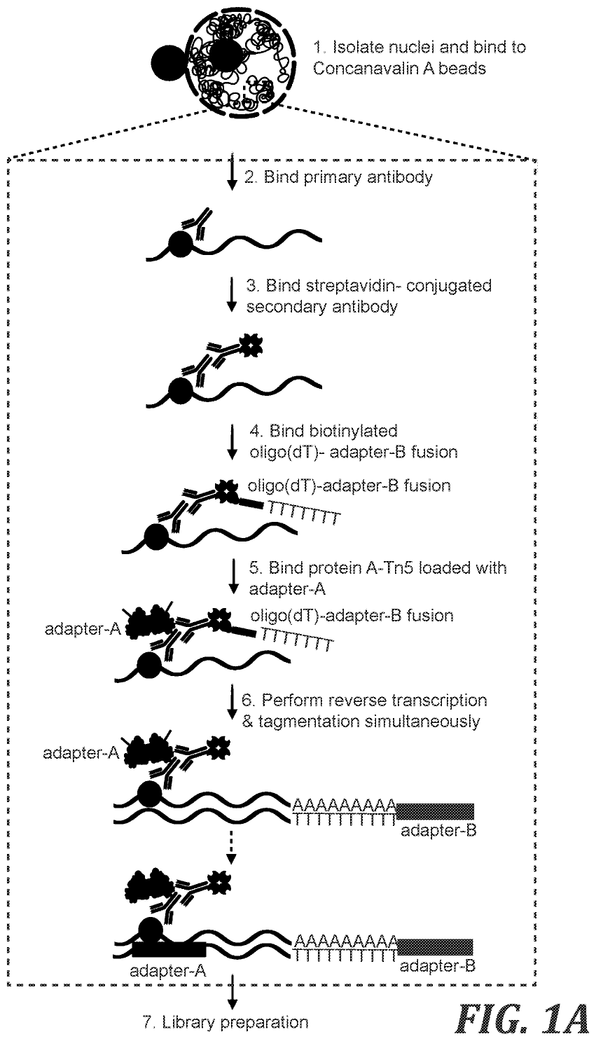

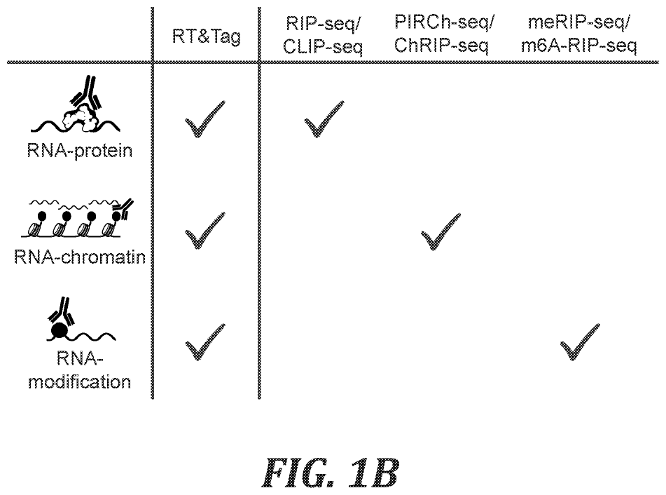

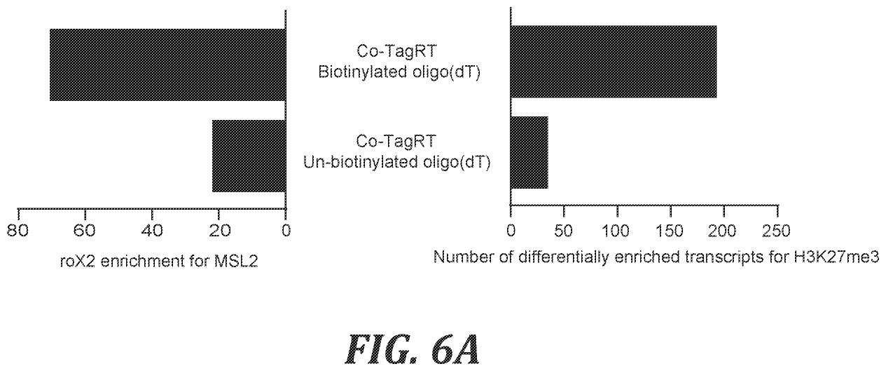

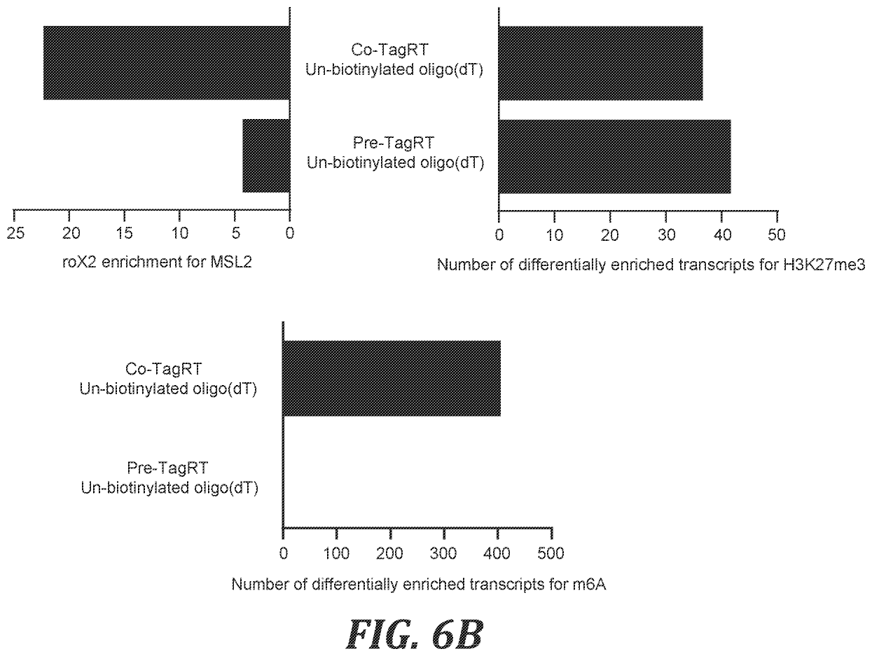

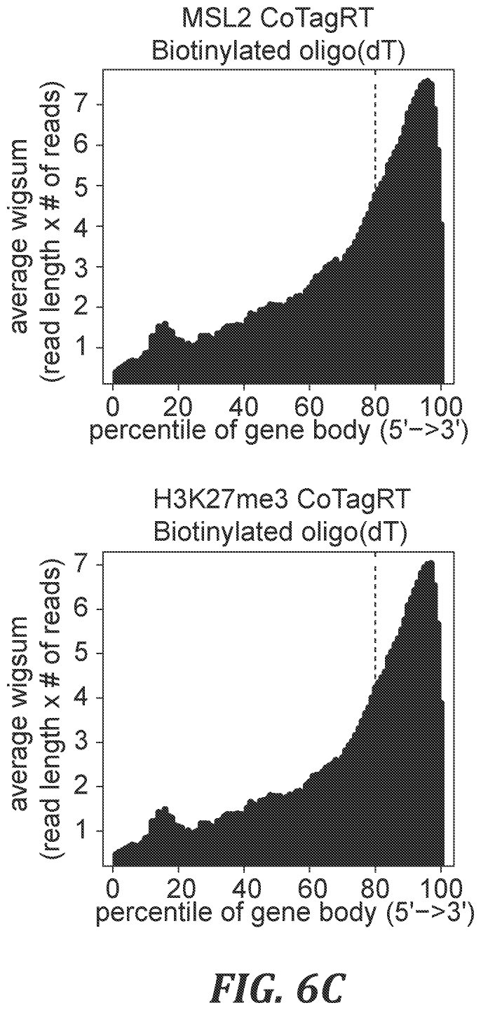

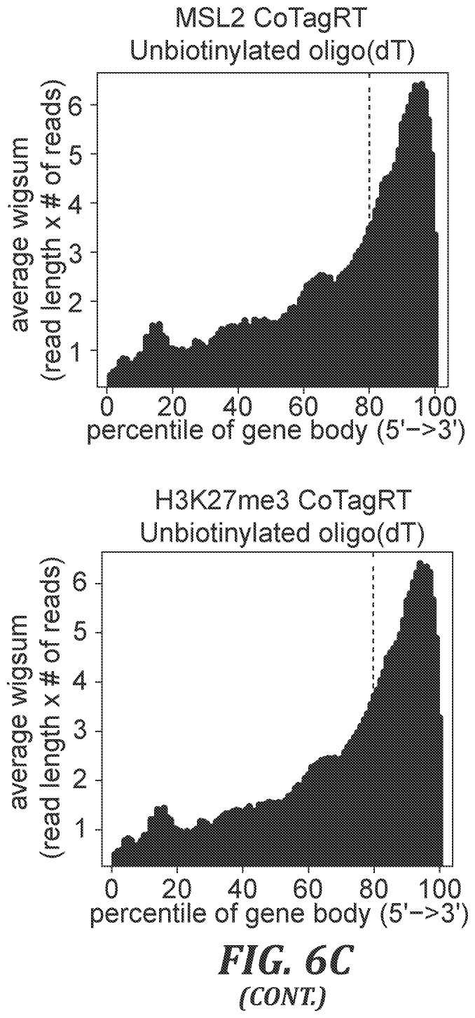

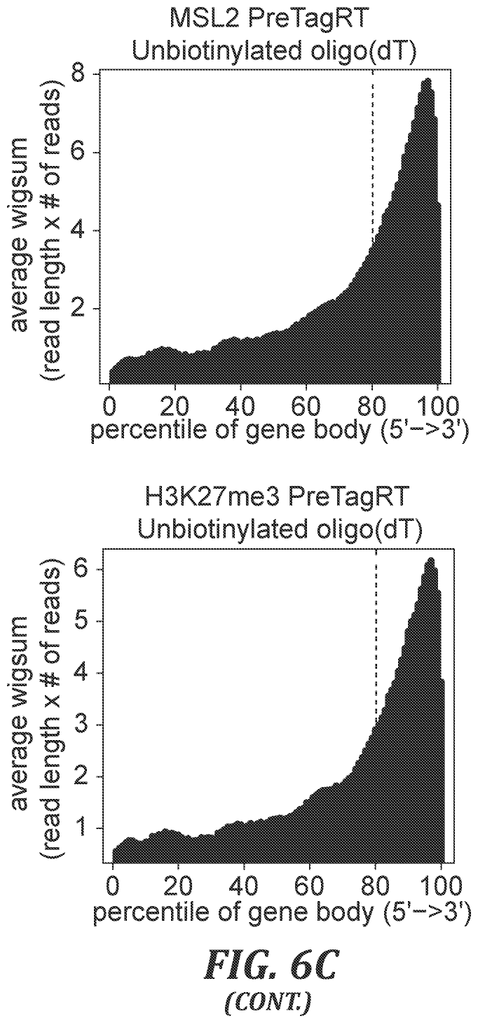

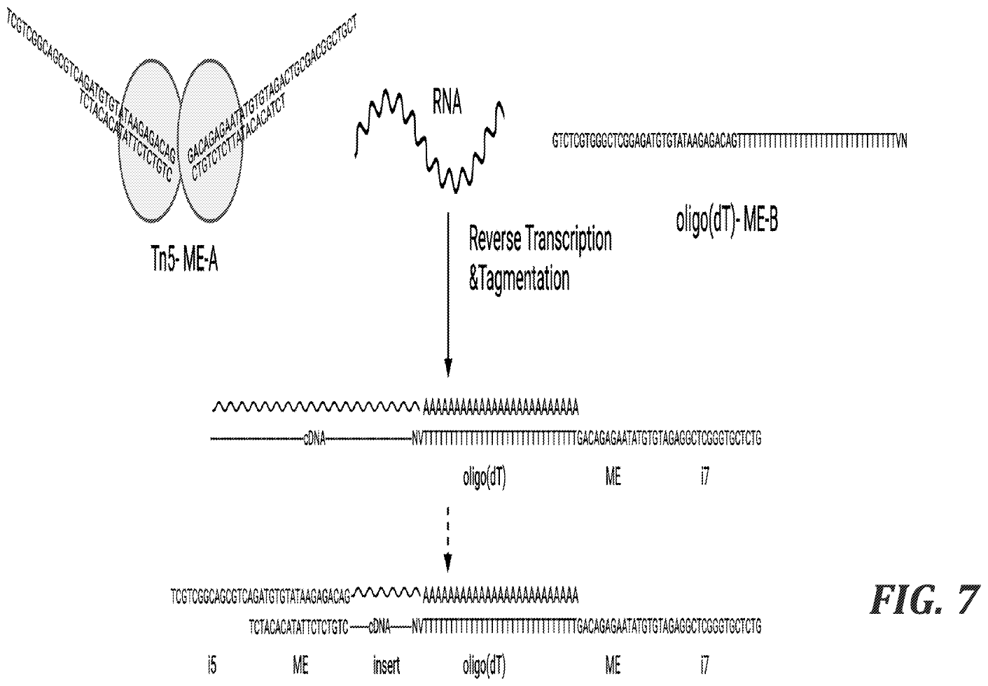

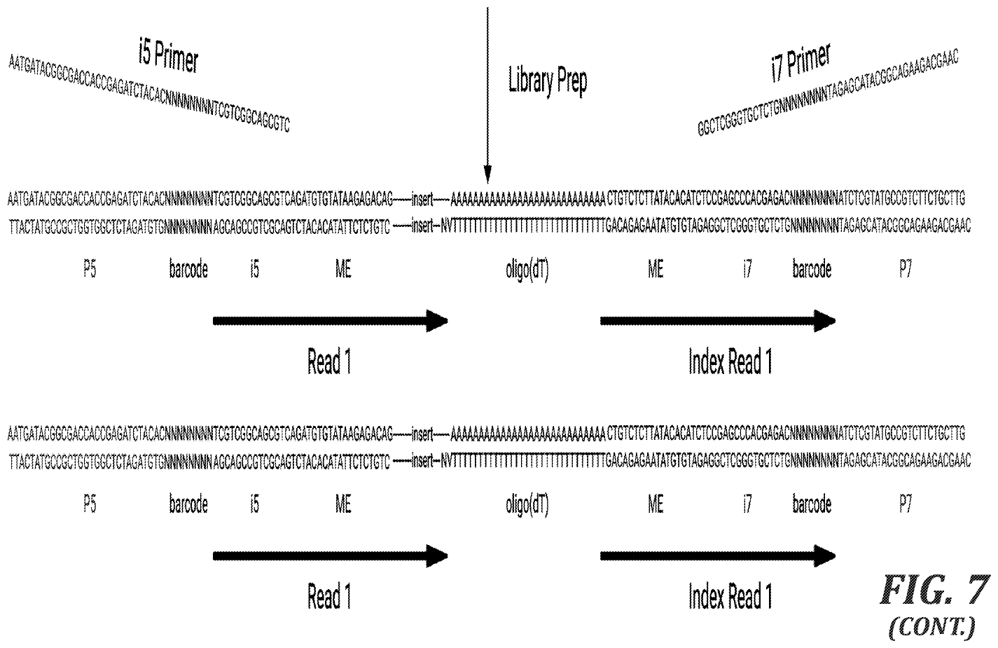

To create a method analogous to CUT&Tag for detecting localized RNAs, the ability of Tn5 to tagment RNA/DNA hybrid duplexes was leveraged (Di, L. et al. RNA sequencing by direct tagmentation of RNA/DNA hybrids. Proc. Natl. Acad. Sci. U.S.A 117, 2886-2893 (2020); Lu, B. et al. Transposase-assisted tagmentation of RNA/DNA hybrid duplexes. Elife 9 (2020)). Briefly, nuclei are first isolated, a factor-specific primary antibody is bound, a streptavidin-conjugated secondary antibody is bound, and then biotinylated oligo(dT)-adapter oligos and pA-Tn5 are tethered to that (FIG. 1A). Using biotinylated oligo(dT)-adaptor fusions increases the signal-to-noise ratio by selectively priming nearby RNA for reverse transcription (RT) (FIG. 6A). Addition of reverse transcriptase then converts mature transcripts near the binding site to RNA/DNA hybrids, which are tagmented by the juxtaposed pA-Tn5. RT and tagmentation are then performed within one incubation step in a compatible buffer. With simultaneous RT and tagmentation, higher transcript enrichment was detected compared with sequential RT and tagmentation (FIG. 6B). This can be attributed to RT altering RNA secondary structure which could then disrupt RNA-protein interactions or mask epitope binding sites. Hence, the simultaneous RT and tagmentation approach can preserve endogenous RNA interactions until the time of tagmentation without sacrificing RT efficiency (FIG. 6C). After RT and tagmentation, the pA-Tn5 is stripped off with SDS and sequencing libraries are amplified using PCR. To generate sequencing libraries only from RNA instead of from genomic DNA, the i7 adaptor sequence is appended 5′ to the oligo(dT) sequence, ensuring its integration into all reverse transcribed transcripts (FIG. 7). The i5 adaptor is loaded into Tn5 and is integrated into RNA-cDNA hybrids via tagmentation. As such, only tagmented RNA-cDNA hybrids have both adaptors necessary for library amplification, whereas genomic DNA lacks the i7 adaptor. With the i7 adaptor appended to the oligo(dT), the amplified libraries should detect signal from the 3′ end of the RNA. This means that only a small segment of the RNA needs to be effectively reverse transcribed to be detected by RT&Tag. Not having to reverse transcribe the entirety of the transcripts minimizes variation arising from RT such as interference with the processivity of the reverse transcriptase due to RNA secondary structure, protein binding and RNA length. To explore the capabilities of RT&Tag, the platform was applied to address diverse problems in RNA-chromatin biology. These include identifying RNA interacting with proteins and chromatin domains and detection of transcripts enriched for post-transcriptional modifications. It was found that all three modalities could be assayed using RT&Tag, unlike immunoprecipitation-based methods which required a different method for each modality (FIG. 1B).

RT&Tag Captures the Interaction Between MSL2 and the roX2 Noncoding RNA

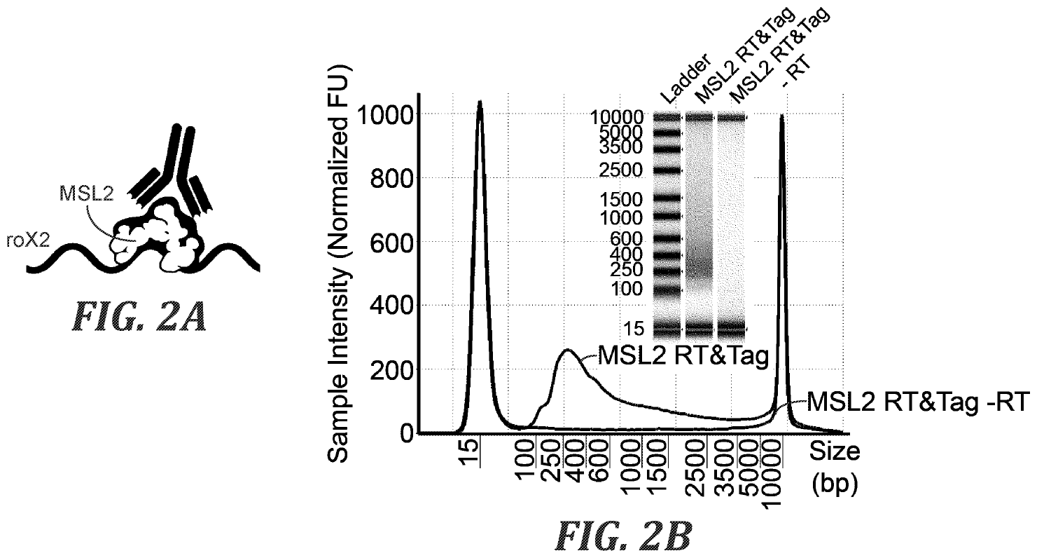

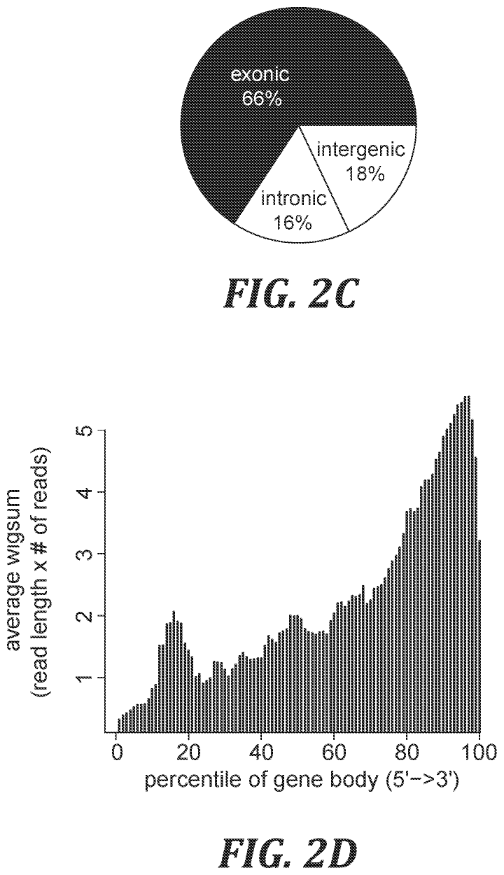

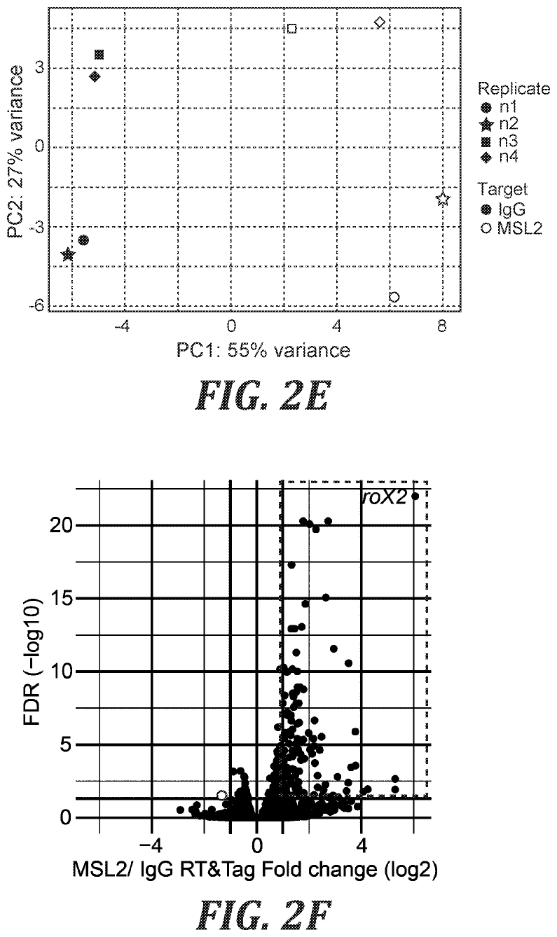

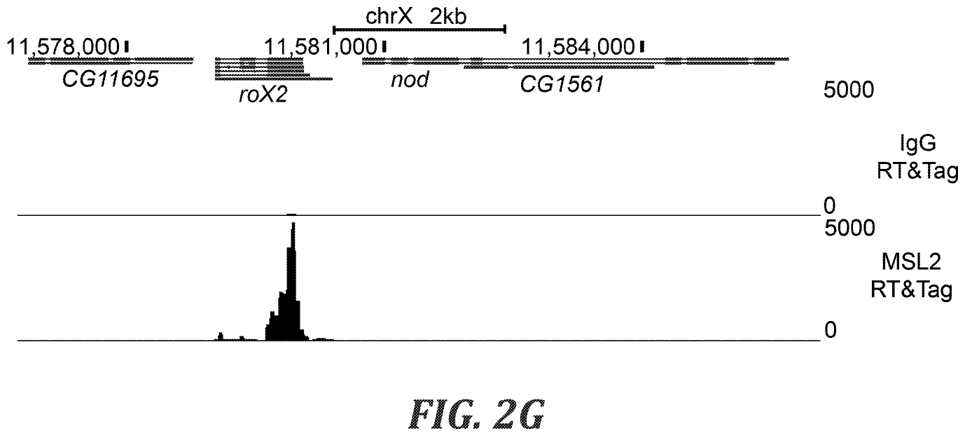

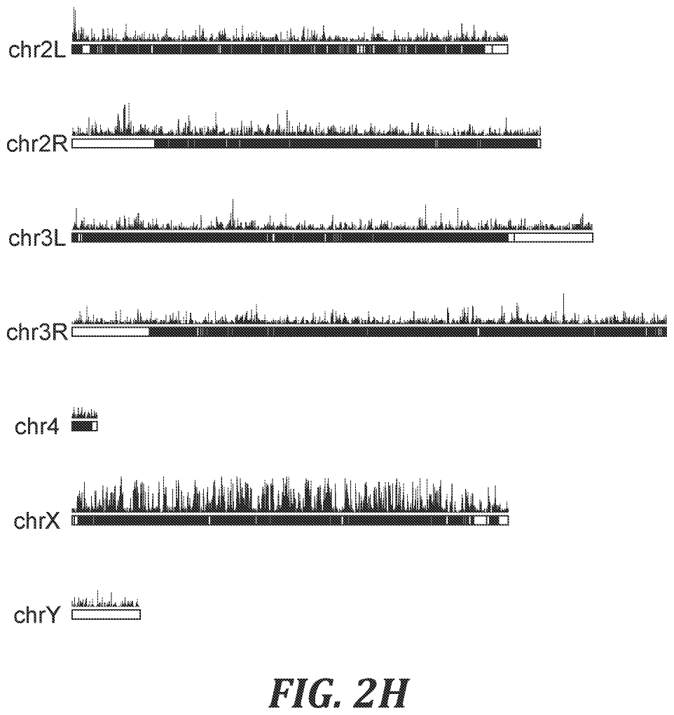

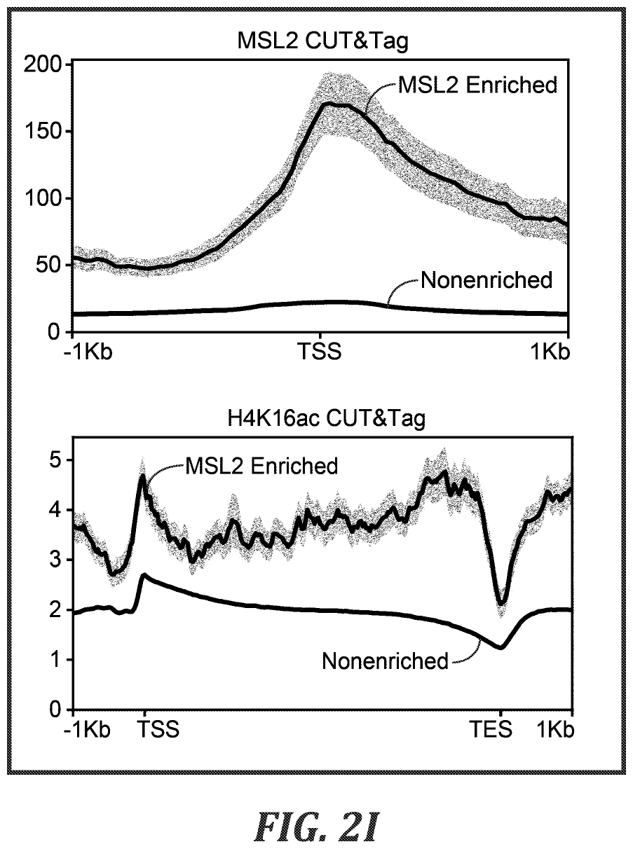

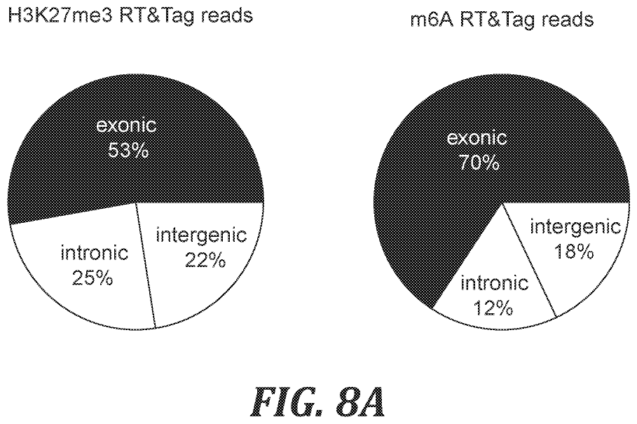

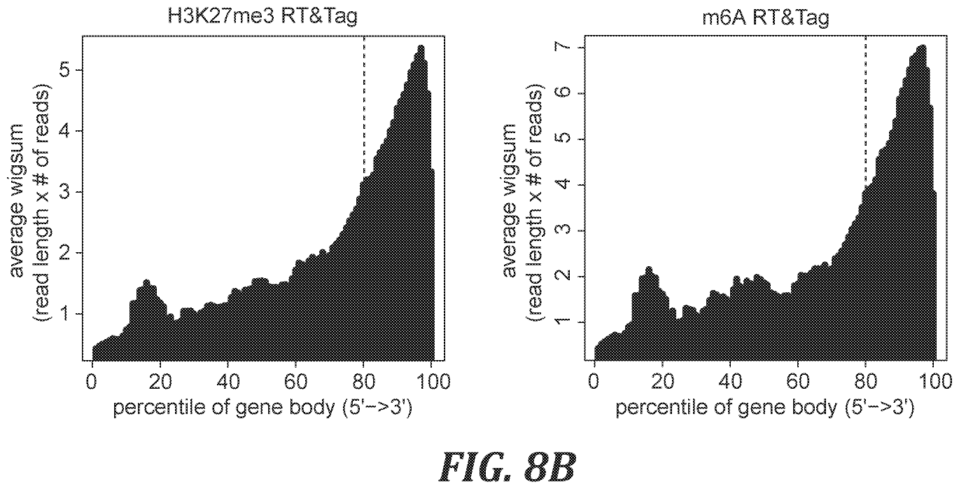

As a proof of concept, antibodies were used to target the RNA-associated dosage compensation complex in the male Drosophila S2 cell line (FIG. 2A). The MSL complex coats the male X chromosome to upregulate gene expression by depositing the activation-associated H4K16ac mark (Conrad, T. & Akhtar, A. Dosage compensation in Drosophila melanogaster: epigenetic fine-tuning of chromosome-wide transcription. Nat. Rev. Genet. 13, 123-134 (2012)). The long non-coding RNA (IncRNA) roX2 is bound by MSL2, an interaction that was expected to be detect using RT&Tag (Conrad, T. et al., Nat. Rev. Genet. 13, 123-134 (2012)). Using an anti-MSL2 antibody, RT&Tag DNA sequencing libraries were generated. Four features indicated that these libraries resulted from tagmentation of reverse transcribed RNA/DNA hybrids. First, no libraries were produced when reverse transcriptase was omitted (FIG. 2B). Second, while CUT&Tag for chromatin targets produced a nucleosomal ladder, RT&Tag libraries had a broad size distribution ranging predominantly from 200 bp to 1000 bp with no nucleosomal pattern (FIG. 2B). Third, mapped RT&Tag reads were primarily of exonic origin (66%) with a small number of intronic (16%) and intergenic reads (18%) (FIG. 2C, FIG. 8A). Finally, reads mostly fell at the 3′ ends of gene bodies consistent with priming from the poly-A tail of mature transcripts by the oligo-dT-adaptor fusion (FIG. 2D, FIG. 8B). Altogether, these findings demonstrate that the RT&Tag signal is exclusively from RNA.

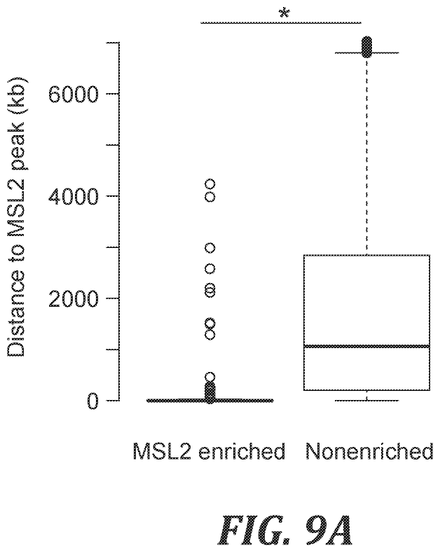

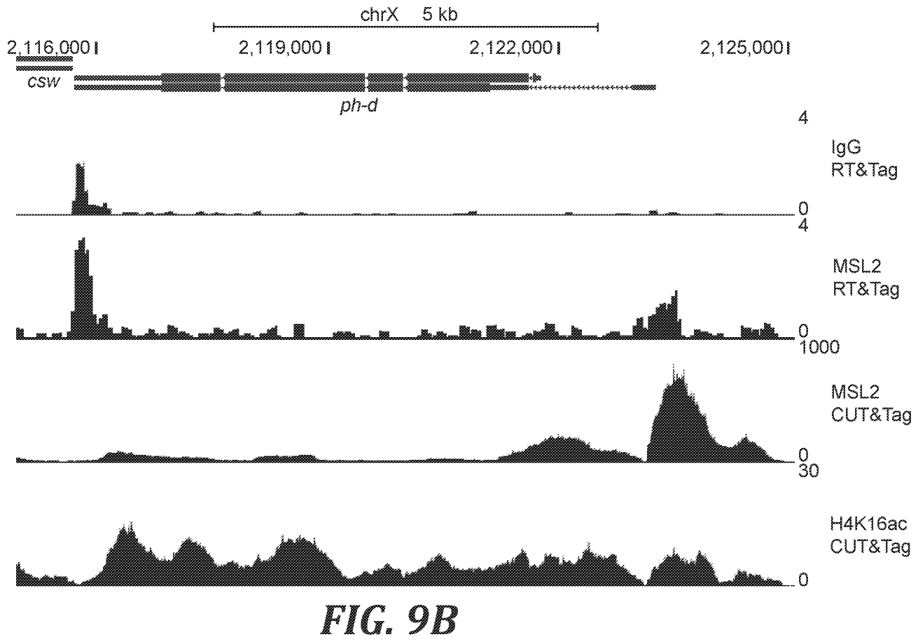

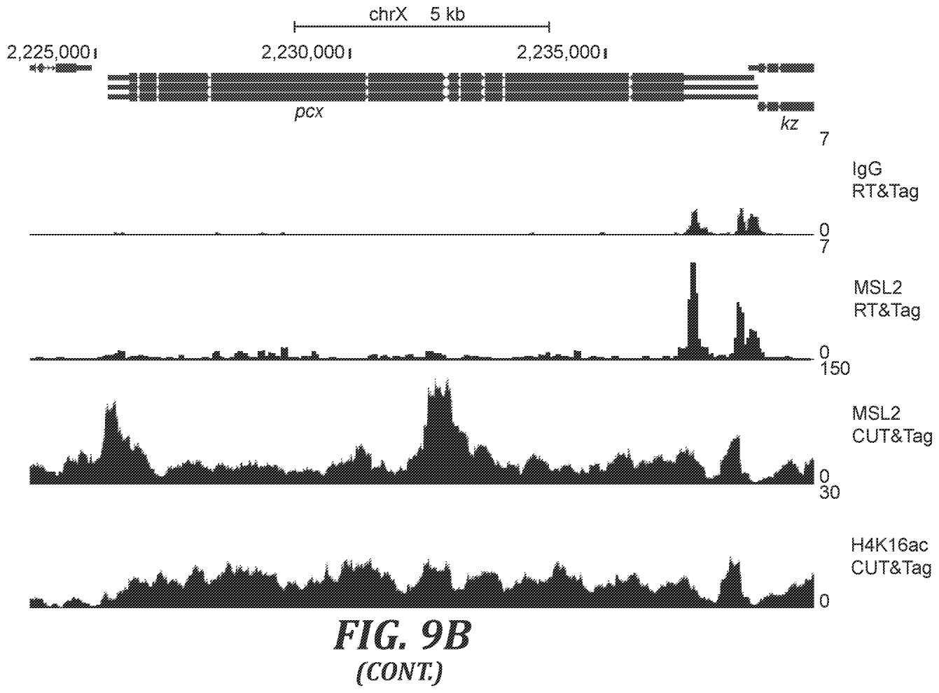

The performance of MSL2 RT&Tag was then evaluated. Differences between MSL2 RT&Tag and the IgG background control were assessed using principal component analysis (PCA) (FIG. 2E). The first principal component captured a clear separation (55% variance) between IgG and MSL2 libraries. This separation was greater than the second principal component which captured the variability between replicates (27% variance). Differential enrichment of MSL2-targeted transcripts over IgG (>2 Fold Change (FC), <0.05 FDR) identified 121 transcripts, of which roX2 showed very high enrichment and statistical significance (67 FC, <1×10⁻²² FDR; FIG. 2F). This enrichment of MSL2 RT&Tag signal over IgG is illustrated over the gene body of roX2 using UCSC genome browser tracks, highlighting a clear 3′ bias in the distribution of reads (FIG. 2G). Apart from roX2, 120 transcripts were differentially enriched for MSL2. The MSL2 RT&Tag signal normalized for IgG showed a strong preference for the X-chromosome (56.3% of >4-fold enriched bins, FIG. 2H). Given that MSL2 binds across the X-chromosome, it was asked whether MSL2 RT&Tag captured RNA that was transcribed proximal to these MSL2 binding sites. Hence, the MSL2 CUT&Tag signal was mapped at the transcriptional start sites (TSSs) of MSL2 enriched or nonenriched transcripts. Additionally, H4K16ac CUT&Tag signal was mapped over the gene bodies of MSL2 enriched or nonenriched transcripts. Higher MSL2 and H4K16ac CUT&Tag signal was observed for MSL2 RT&Tag enriched than nonenriched transcripts (FIG. 2I). Furthermore, 75% of MSL2-enriched transcripts were within 13 kb of an MSL2 binding peak which is much closer than for nonenriched transcripts (12,608 bp vs 2,841,851 bp, p<2.2×10⁻¹⁶, FIG. 9A). As an example, MSL2 and H4K16ac CUT&Tag signal can be seen over the gene bodies of MSL2 RT&Tag enriched transcripts, ph-d and pcx (FIG. 9B). Overall, these results show that RT&Tag recapitulates the well-known MSL2-roX2 interaction and captures interactions between MSL2 and transcripts found within its vicinity. Distinguishing direct versus proximal interactions can be guided by the idea that proximity interactions should be transient and result in weaker enrichment. As such, enrichment for roX2 is a unique outlier both in fold change and FDR while the proximal transcripts found on the X-chromosome exhibit either low fold change or low FDR.

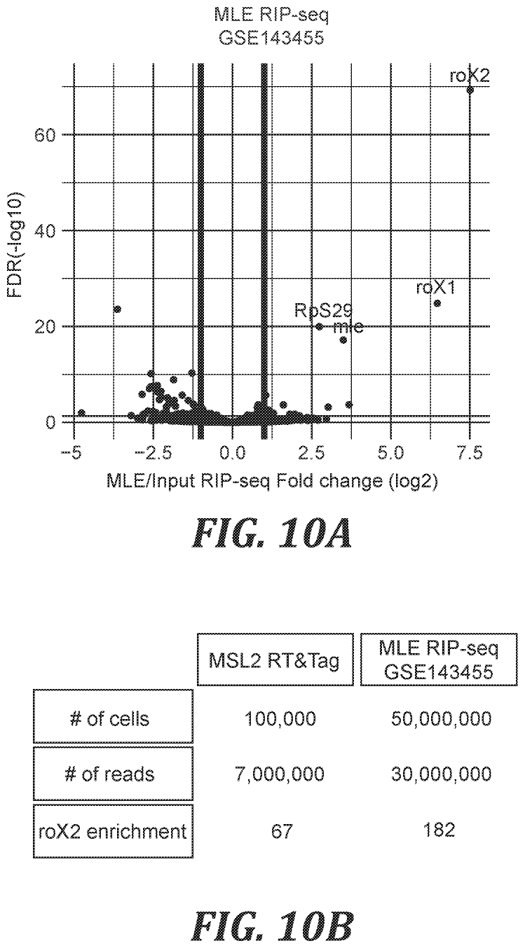

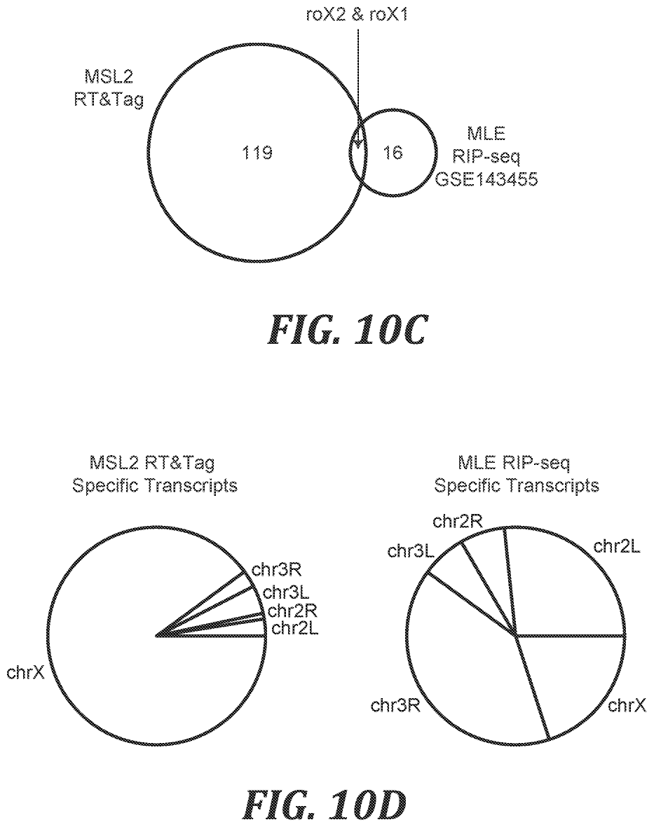

The MSL2 RT&Tag data was then compared to a published RIP-seq dataset, which targeted a subunit of the Drosophila MSL complex, maleless (ILE). Like RT&Tag, MILE RIP-seq was able to identify the interaction between MLE and roX2 in S2 cells (FIG. 10A). However, to achieve a comparable degree of enrichment for roX2, RIP-seq required 500 times the number of cells and 4 times as many sequencing reads as RT&Tag (FIG. 10B). Apart from the roX RNAs, RT&Tag and RIP-seq picked up transcripts that were unique to each method (FIG. 10C). Transcripts unique to RT&Tag were predominantly transcribed from the X-chromosome unlike the transcripts unique to RIP-seq (FIG. 10D). This comparison highlights the fundamental difference between RT&Tag and immunoprecipitation-based methods. Being a proximity labelling technique, RT&Tag can pick up transcripts near MSL complex binding sites, whereas RIP-seq captures binding interactions within cell lysates, some of which might not occur under endogenous conditions.

RT&Tag Captures Transcripts within Polycomb Domains

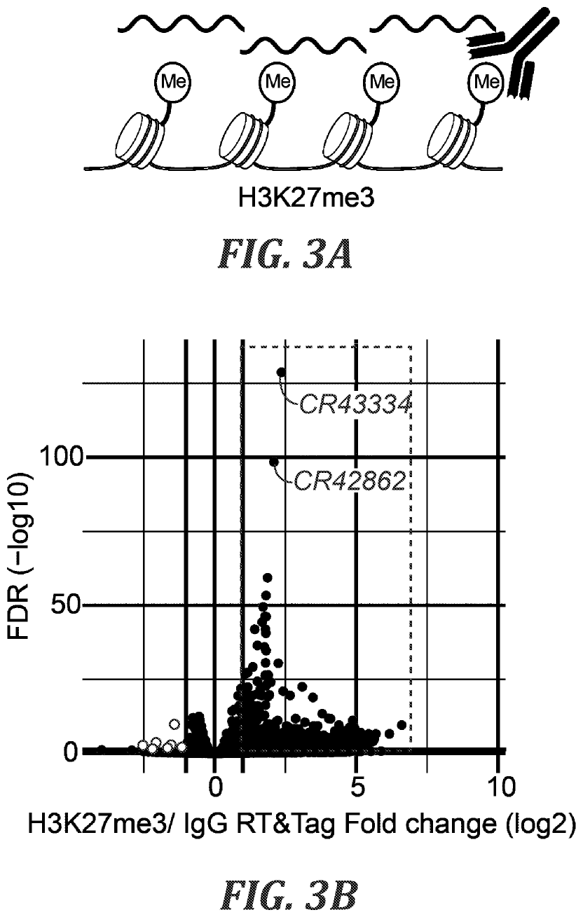

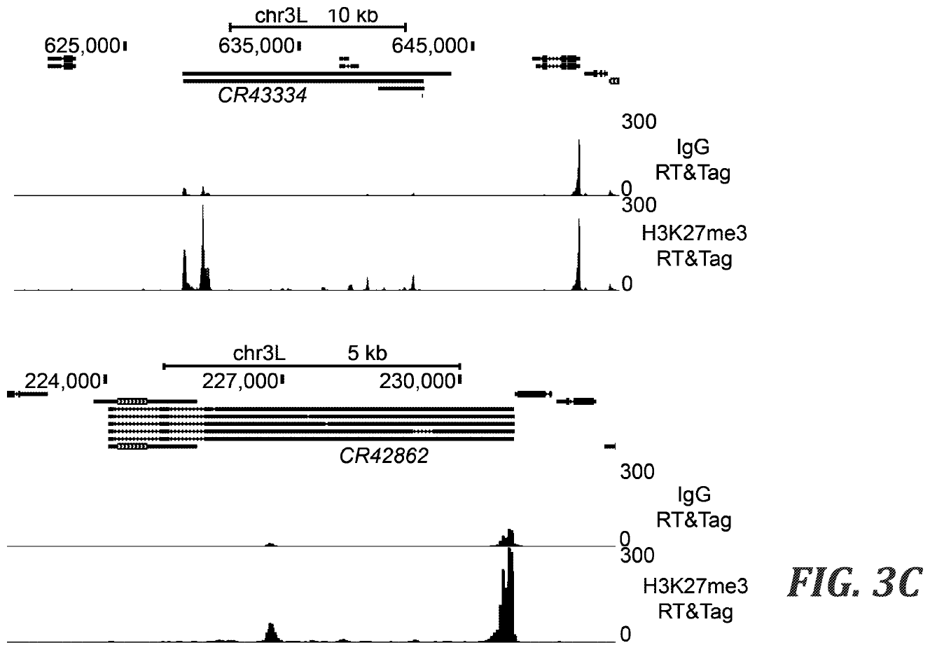

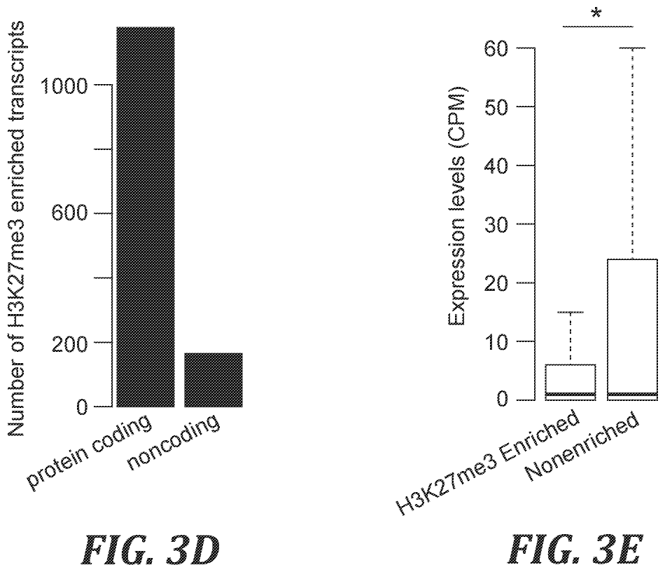

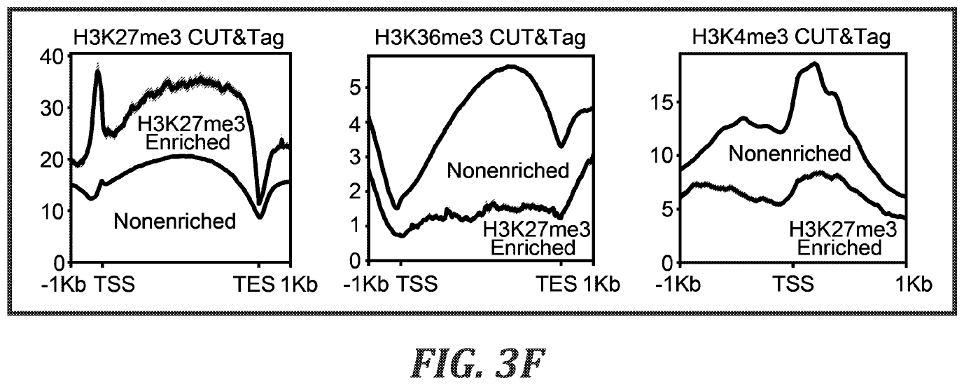

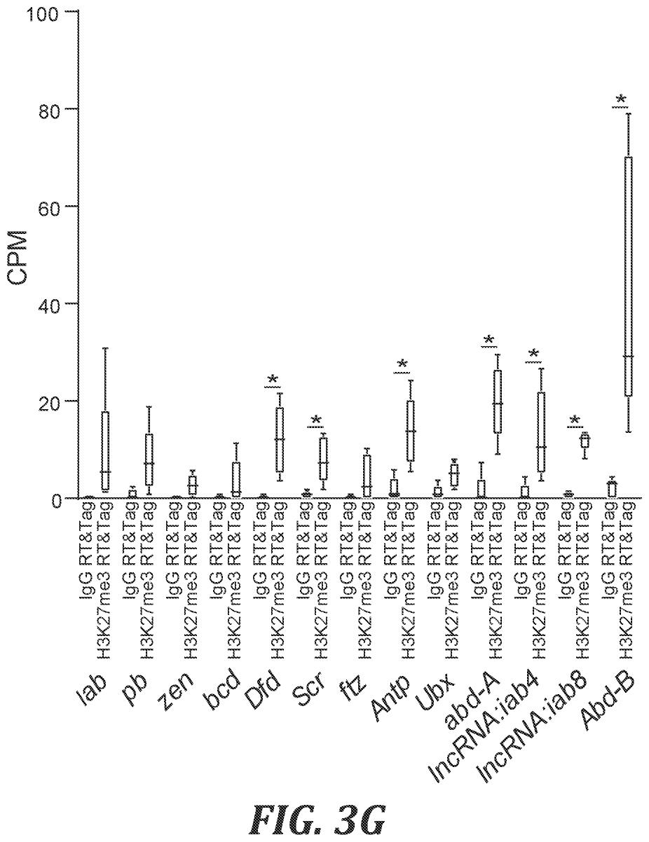

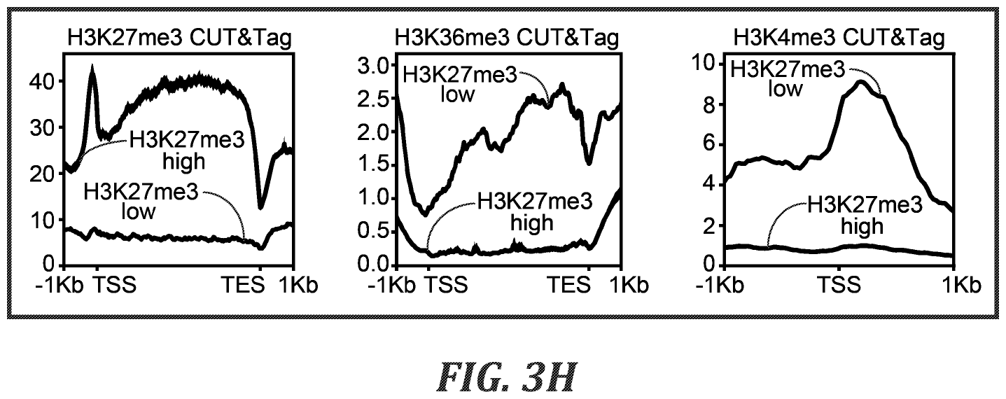

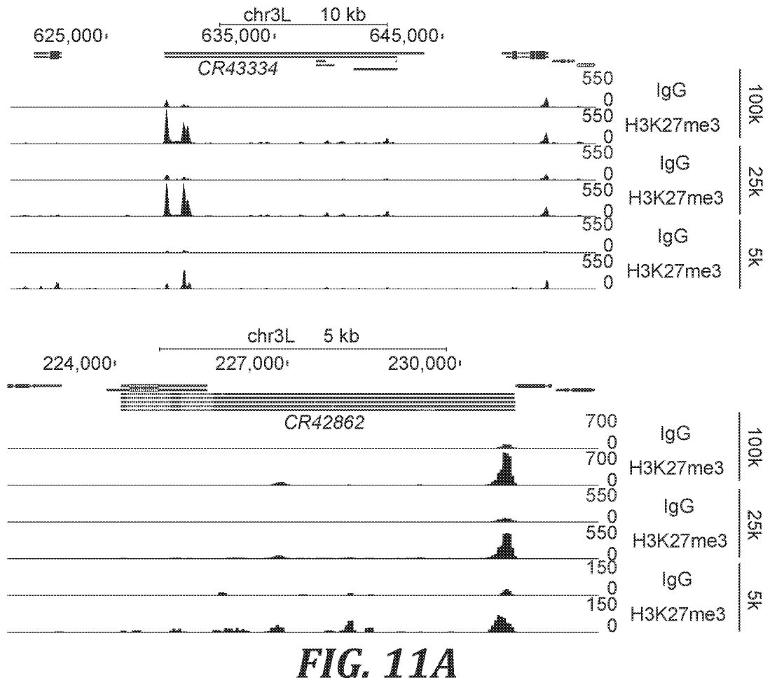

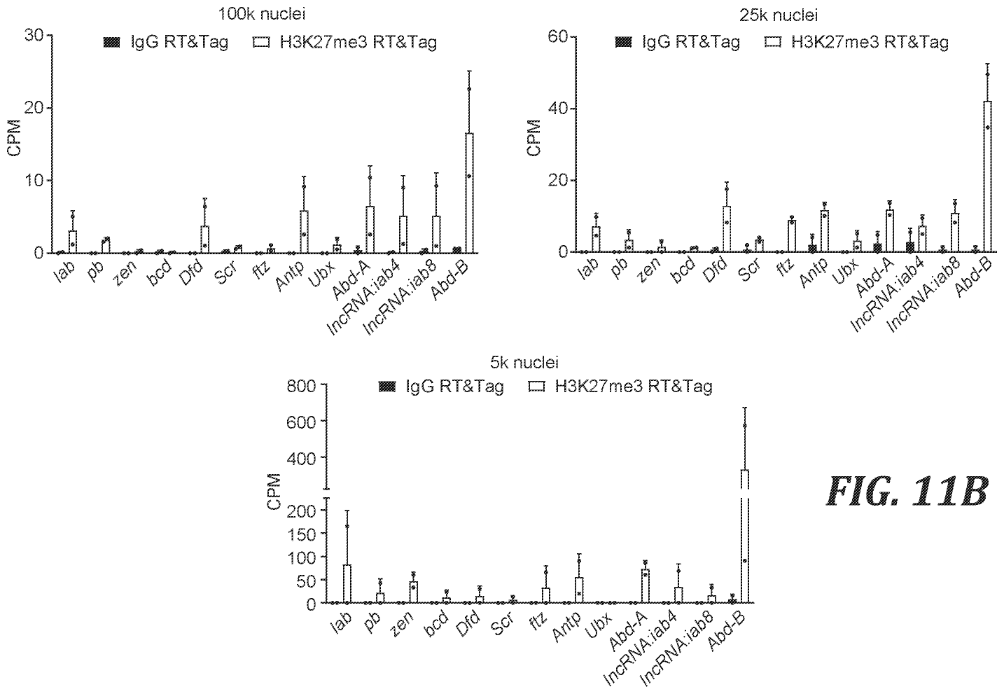

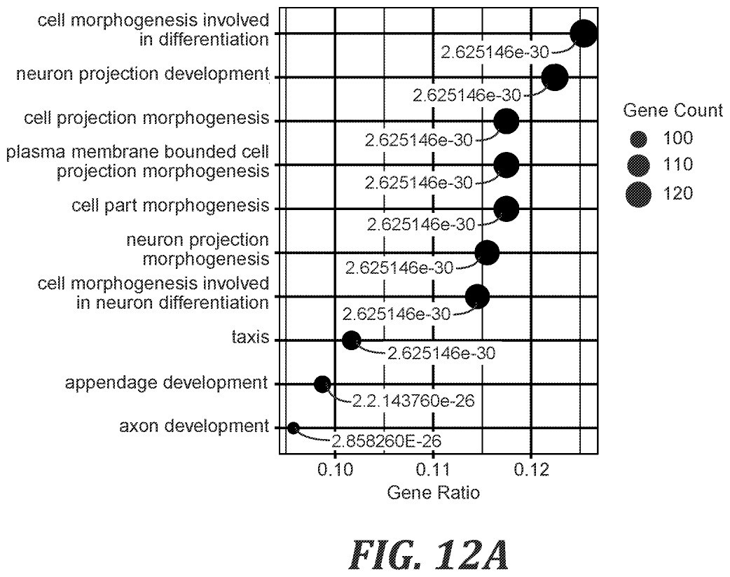

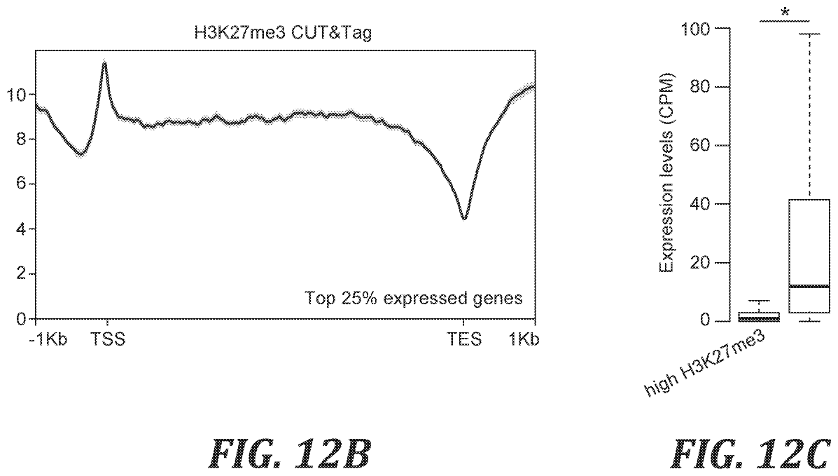

After validating RT&Tag using MSL2, RT&Tag was applied to identify RNA associated with chromatin domains (FIG. 3A). Polycomb domains are large regions of chromatin decorated with repressive histone H3K27me3 marks (Cheutin, T. & Cavalli, G. The multiscale effects of polycomb mechanisms on 3D chromatin folding. Crit. Rev. Biochem. Mol. Biol. 54, 399-417 (2019); Blackledge, N. P. & Klose, R. J. The molecular principles of gene regulation by Polycomb repressive complexes. Nat. Rev. Mol. Cell. Biol. 22, 815-833 (2021)). They make for an appealing target as studies in mammals have implicated RNA in their establishment and maintenance (Blackledge, N. P. & Klose, R. J. The molecular principles of gene regulation by Polycomb repressive complexes. Nat. Rev. Mol. Cell. Biol. 22, 815-833 (2021)). Targeting H3K27me3 with an antibody, RT&Tag identified 1342 transcripts that are differentially enriched for H3K27me3 over IgG background (>2 FC, <0.05 FDR; FIG. 3B). As examples, the H3K27me3-targeted RT&Tag signals are shown over the two most statistically significant hits, the IncRNAs CR43334 and CR42862 (FIG. 3C). The performance of H3K27me3 RT&Tag was then assessed with decreasing numbers of input nuclei. The H3K27me3 RT&Tag signal was highly reproducible using 100,000 and 25,000 nuclei and even 5,000 nuclei for CR43334 and CR42862 (FIG. 11A). Next, H3K27me3-enriched transcripts were characterized and were found to be predominantly protein coding (1178 out of 1342) with low expression levels (mean 16.6 counts per million (CPM) vs 97.1 CPM for nonenriched genes, p<2.2×10⁻¹⁶) (FIG. 3D, E). Additionally, H3K27me3 RT&Tag-enriched transcripts had more repressive H3K27me3 CUT&Tag signal and lower active H3K36me3 and H3K4me3 CUT&Tag signal at their TSS or over their gene bodies than nonenriched transcripts (FIG. 3F). In line with this, K3K27me3 RT&Tag-enriched transcripts enriched for GO terms associated with developmental biological processes, which are associated with Polycomb (Lee, T. I. et al. Control of developmental regulators by Polycomb in human embryonic stem cells. Cell 125, 301-313 (2006)) (FIG. 12A). Altogether, these data suggest that H3K27me3 RT&Tag-enriched transcripts are from repressed genes within Polycomb domains. These include classic examples of Polycomb repressed genes such as the Hox genes (Kassis, J. A., Kennison, J. A. & Tamkun, J. W. Polycomb and Trithorax Group Genes in Drosophila. Genetics 206, 1699-1725, doi:10.1534/genetics.115.185116 (2017)), which has been found to show strong enrichment for H3K27me3-targeted RT&Tag signal (FIG. 3G, FIG. 11B).

Next, the proportion of H3K27me3-targeted RT&Tag transcripts that were transcribed from regions decorated by H3K27me3 marks was assessed. First, the H3K27me3 CUT&Tag background level cut-off was established in S2 cells as the H3K27me3 CUT&Tag signal over the gene bodies for the top 25% expressed genes (>17 CPM) (FIG. 12B). Using this cut-off, 84.5% (1134 out of 1342) of H3K27me3-RT&Tag enriched transcripts were found to be from regions with substantial H3K27me3 CUT&Tag signal (FIG. 3H). These genes also show low levels of active H3K36me3 and H3K4me3 CUT&Tag signal (FIG. 3H). The remaining 208 H3K27me3-directed RT&Tag enriched transcripts are from outside of H3K27me3 marked regions and show high H3K36me3 and H3K4me3 CUT&Tag signals. These 208 H3K27me3 RT&Tag-enriched genes are more highly expressed than those from H3K27me3 marked regions (mean 60.9 vs 8.5 CPM, p<0.004; FIG. 12C). Given that transcripts captured by RT&Tag must have poly(A) tails, the findings are consistent with the low production of new transcripts from silenced regions, and the subsequent capture of these transcripts near their sites of transcription (Bell, J. C. et al. Chromatin-associated RNA sequencing (ChAR-seq) maps genome-wide RNA-to-DNA contacts. Elife 7 (2018); Li, X. et al. GRID-seq reveals the global RNA-chromatin interactome. Nat. Biotechnol. 35, 940-950 (2017)).

### RT&Tag Captures Transcripts Enriched for the m6A Post-Transcriptional Modification

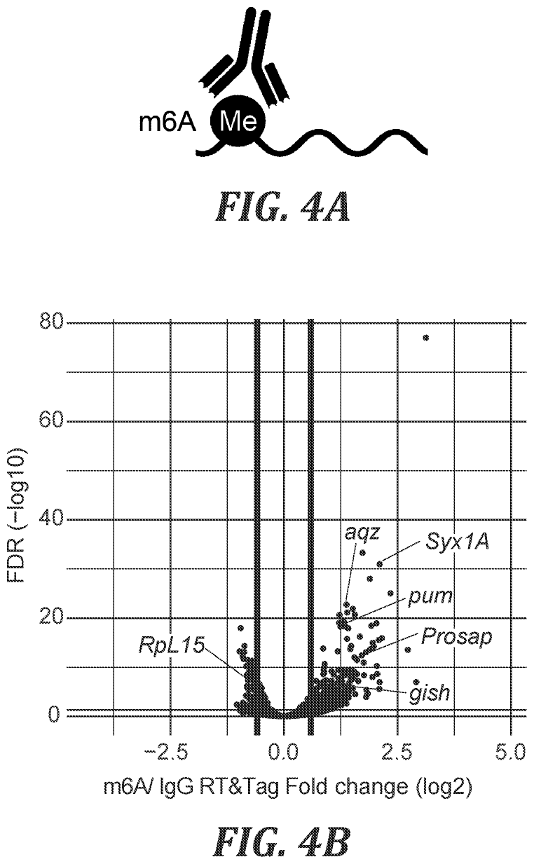

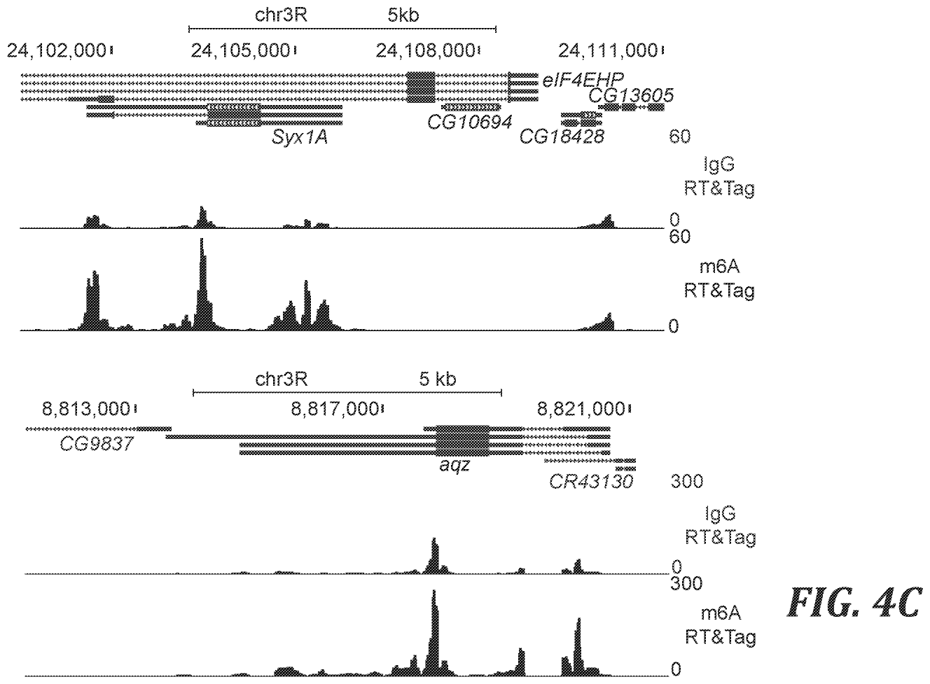

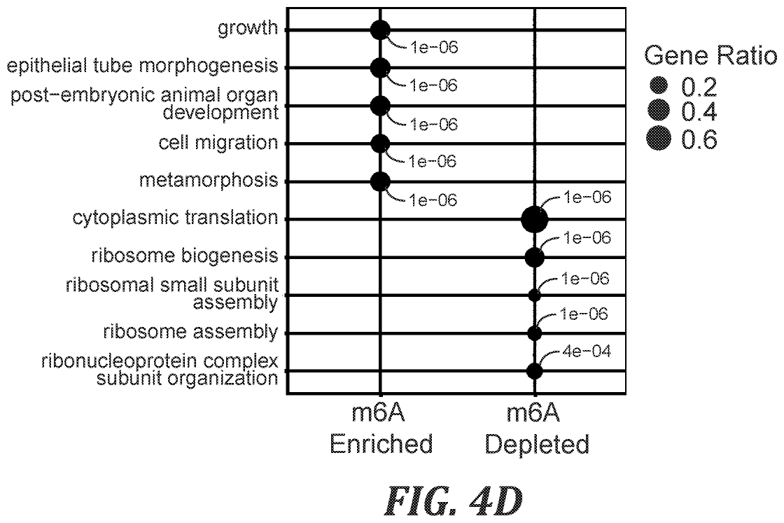

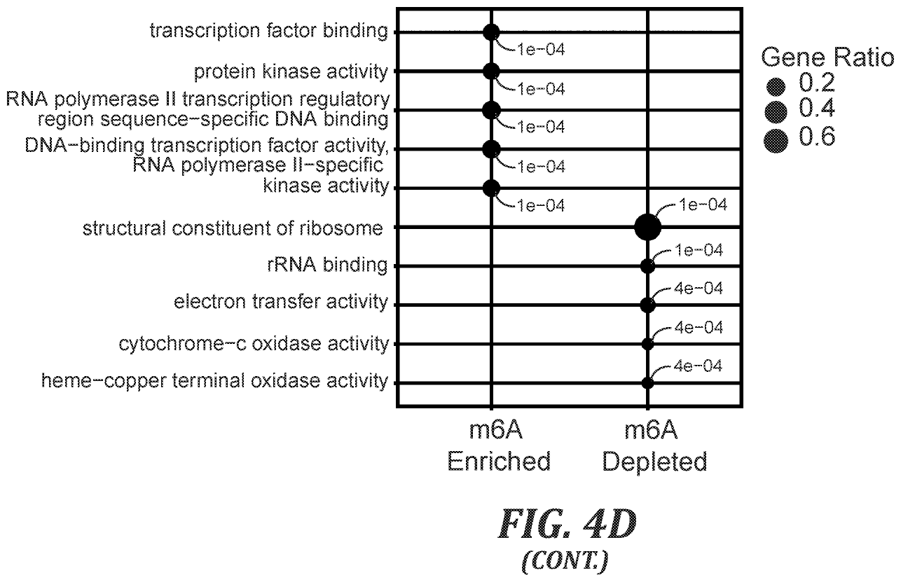

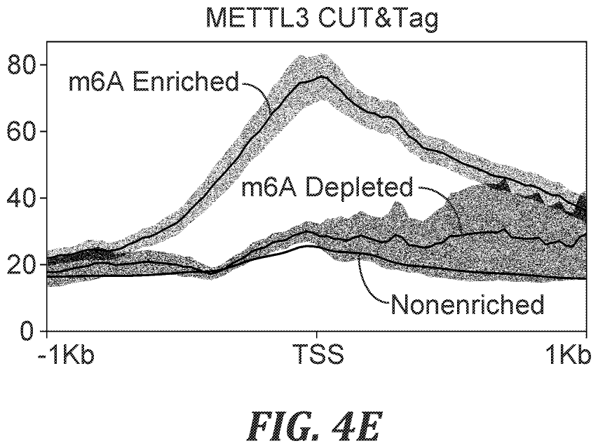

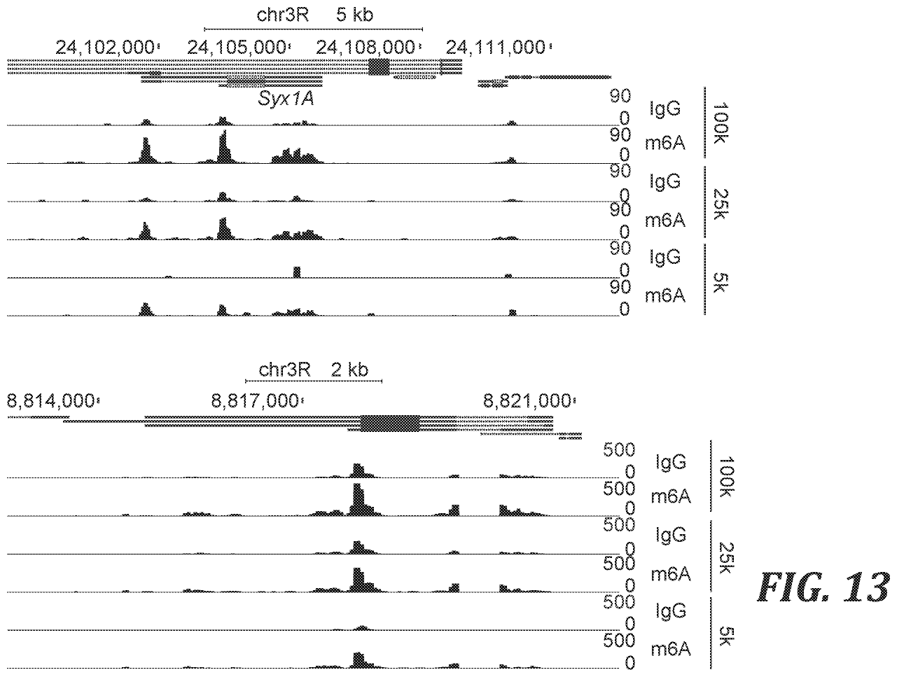

Having demonstrated that RT&Tag can detect RNAs in protein complexes, and chromatin domains, it was tested whether the method could be used for RNA modifications. N6-Methyladenosine (m6A) is the most abundant mRNA post-transcriptional modification and has been implicated in numerous aspects of RNA metabolism (He, P. C. & He, C. m(6) A RNA methylation: from mechanisms to therapeutic potential. EMBO J 40, e105977 (2021)). Commercial antibodies targeting m6A are available and have been used in RNA immunoprecipitation-based methods (i.e., MeRIP-seq and m6A-seq) (Dominissini, D. et al. Topology of the human and mouse m6A RNA methylomes revealed by m6A-seq. Nature 485, 201-206 (2012); Meyer, K. D. et al. Comprehensive analysis of mRNA methylation reveals enrichment in 3′ UTRs and near stop codons. Cell 149, 1635-1646 (2012)). Although these techniques are valuable for pinpointing the location of m6A modifications, they require large amounts of input material and suffer from low reproducibility (McIntyre, A. B. R. et al. Limits in the detection of m(6)A changes using MeRIP/m(6)A-seq. Sci Rep 10, 6590, doi:10.1038/s41598-020-63355-3 (2020)). It was reasoned that RT&Tag could provide insights into whether a particular transcript is enriched or depleted for m6A relative to IgG input control (FIG. 4A). Using RT&Tag, 281 transcripts enriched for m6A (>1.5 FC, <0.05 FDR) and 106 transcripts depleted for this modification were identified (>1.5 FC, <0.05 FDR; FIG. 4B). Of these, aqz, Syx1A, gish, pum and Prosap transcripts have been previously reported as enriched for m6A (Kan, L. et al. A neural m(6)A/Ythdf pathway is required for learning and memory in Drosophila. Nat. Commun. 12, 1458 (2021); FIG. 4B, C). Next, the performance of m6A RT&Tag was assessed with varying numbers of input nuclei. The m6A RT&Tag signal was highly reproducible using 100,000 and 25,000 nuclei and even 5,000 nuclei for aqz and Syx1A (FIG. 13). Transcripts enriched for m6A are associated with development and transcription factor binding Gene Ontology (GO) terms, whereas transcripts depleted for m6A tend to be associated with housekeeping GO terms, especially translational components and processes (FIG. 4D).

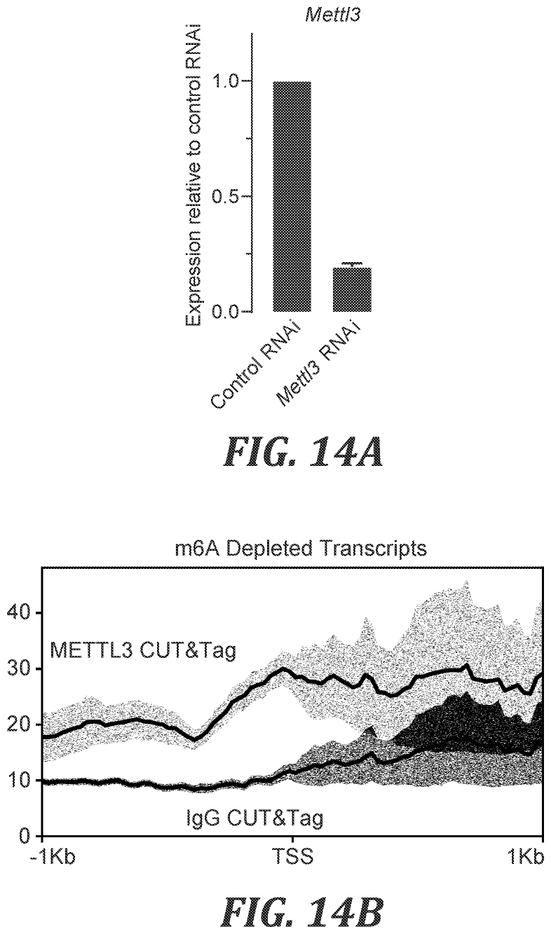

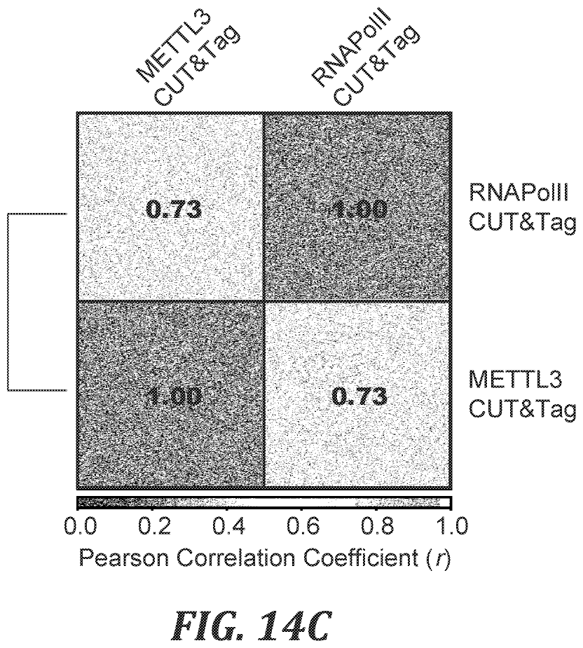

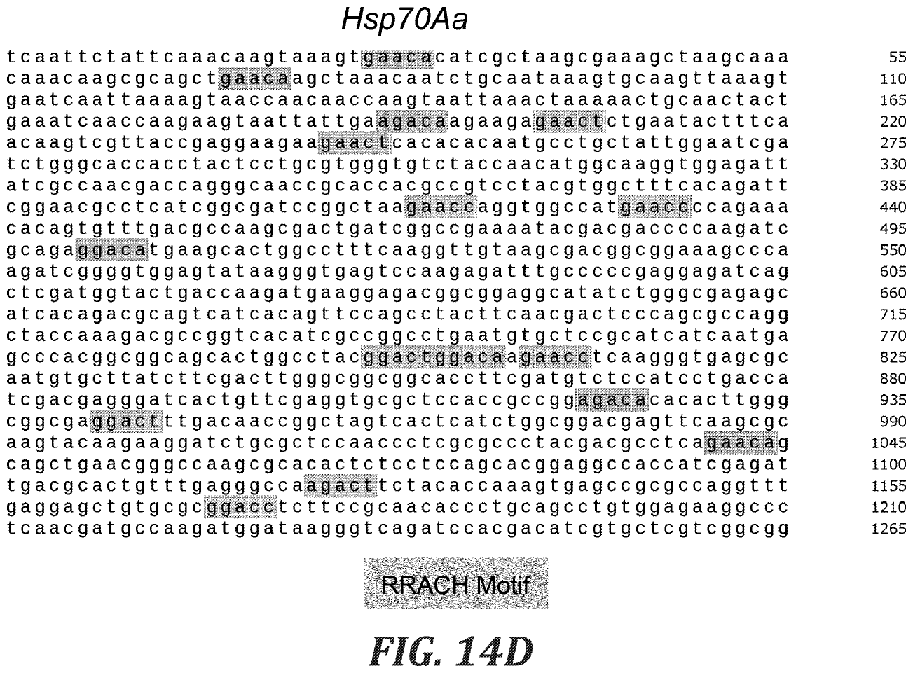

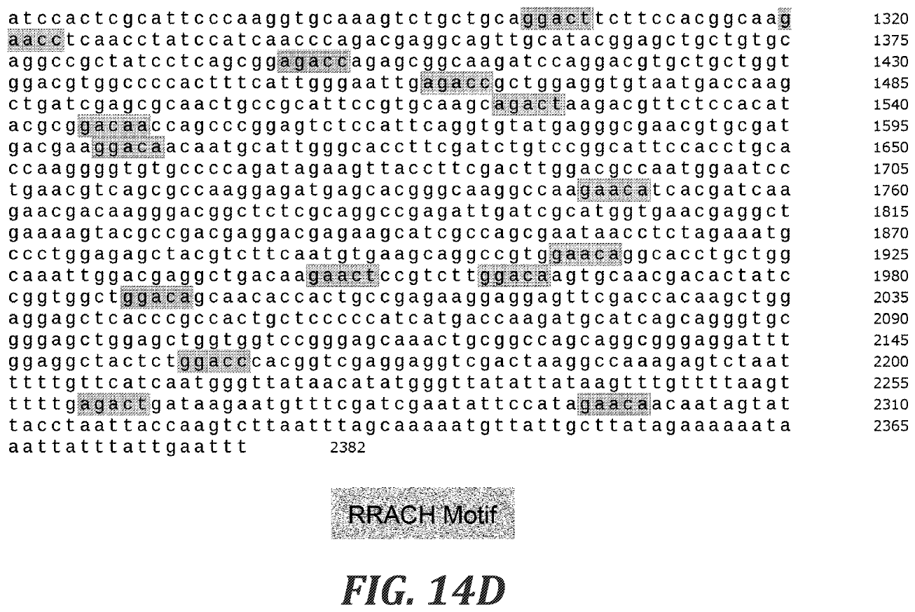

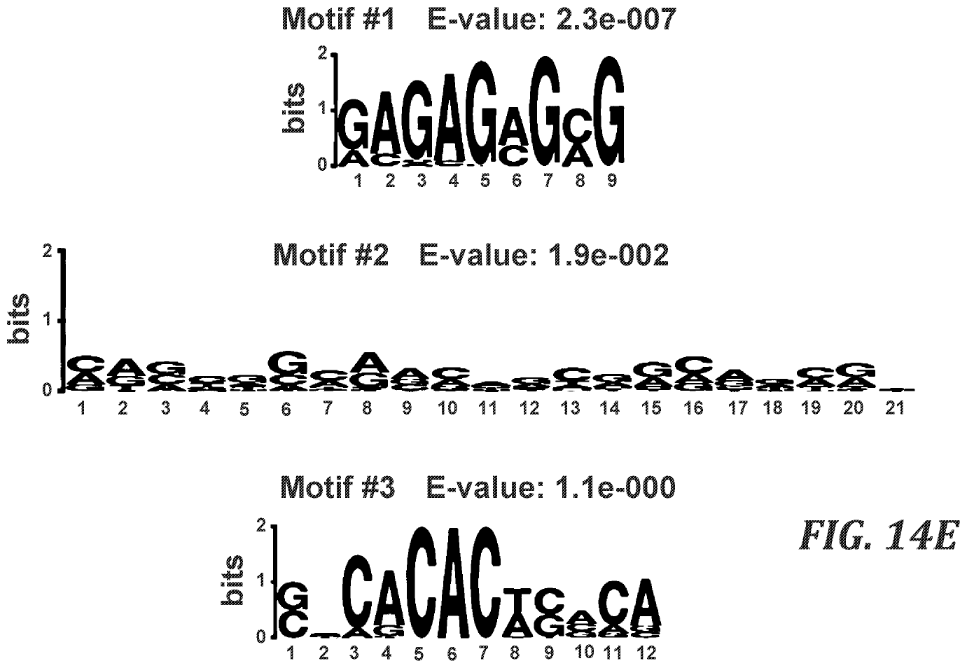

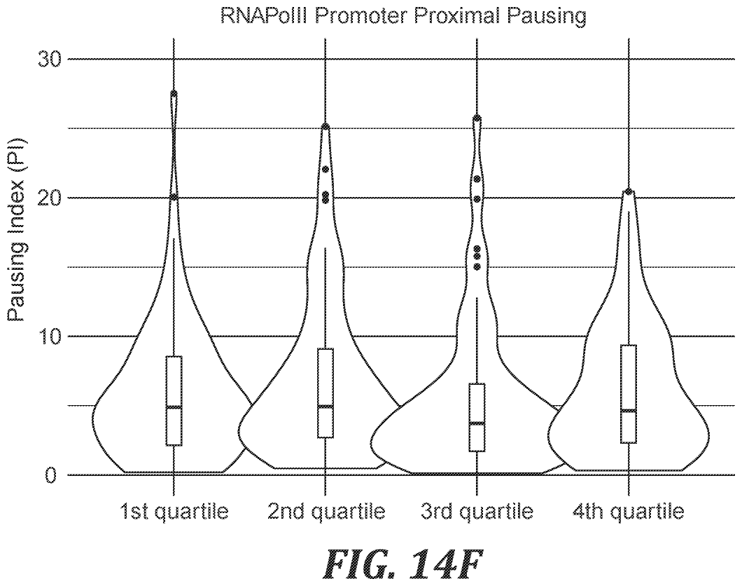

The Drosophila homologue of the METTL3 methyltransferase binds to chromatin and catalyzes the m6A modification on nascent transcripts (Lence, T., Soller, M. & Roignant, J. Y. A fly view on the roles and mechanisms of the m(6)A mRNA modification and its players. RNA Biol. 14, 1232-1240 (2017)). High levels of METTL3 CUT&Tag signal were observed at the TSSs of m6A enriched genes, relative to nonenriched or m6A depleted genes (FIG. 4E). To validate the list of m6A enriched genes, the gene encoding METTL3 (Mettl3, formerly called Inducer of meiosis in yeast or Ime4) was knocked down levels by 80% using RNAi (FIG. 14A). Doing so resulted in a modest decrease (>10%) for 81% of m6A enriched transcripts (FIG. 4F). Altogether, these results show that m6A enriched transcripts identified by RT&Tag are METTL3 methylation dependent.

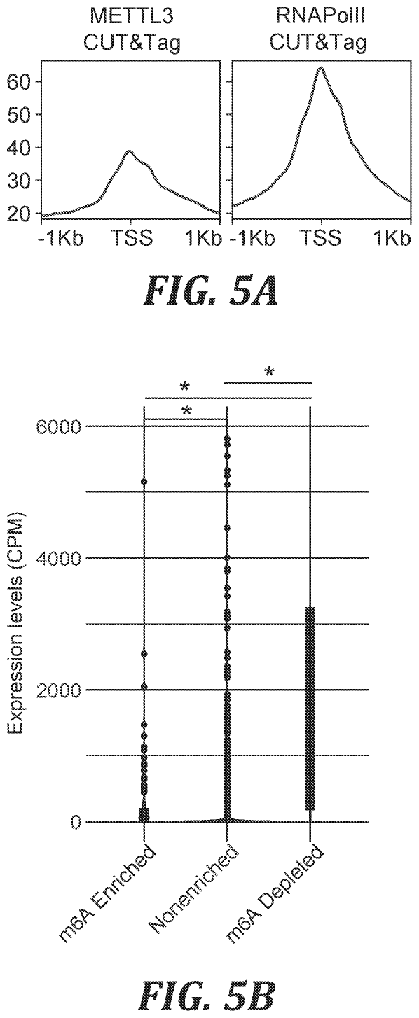

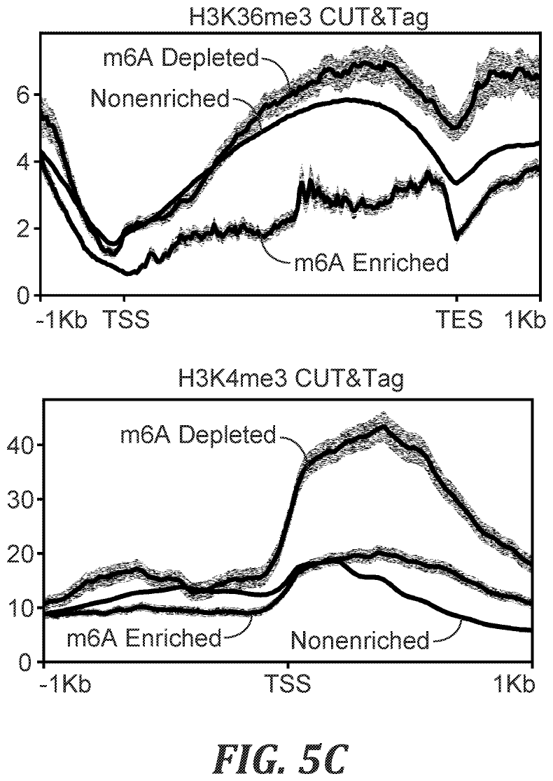

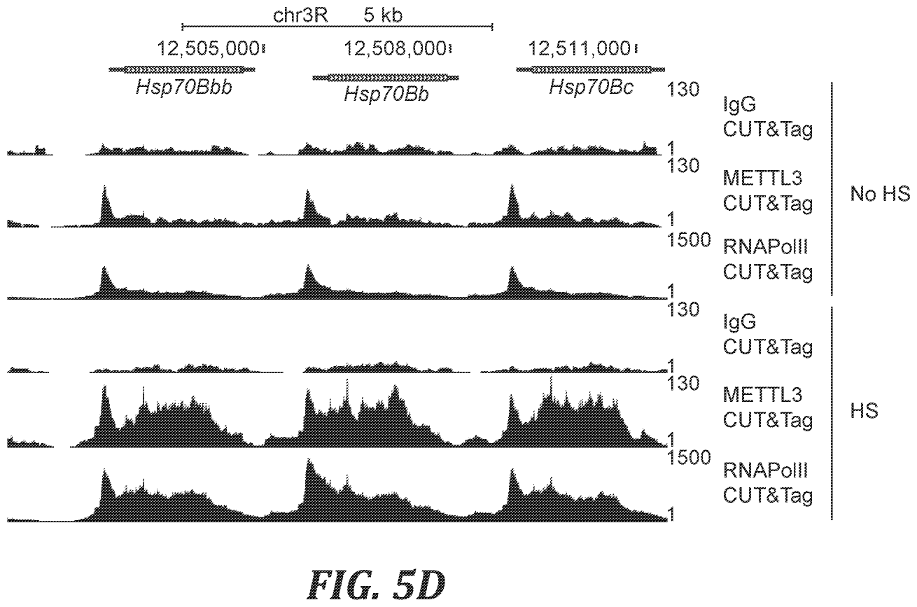

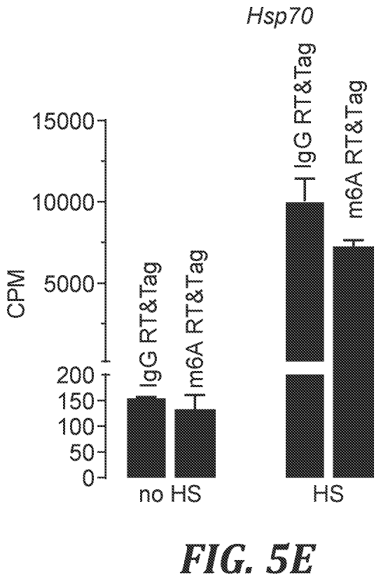

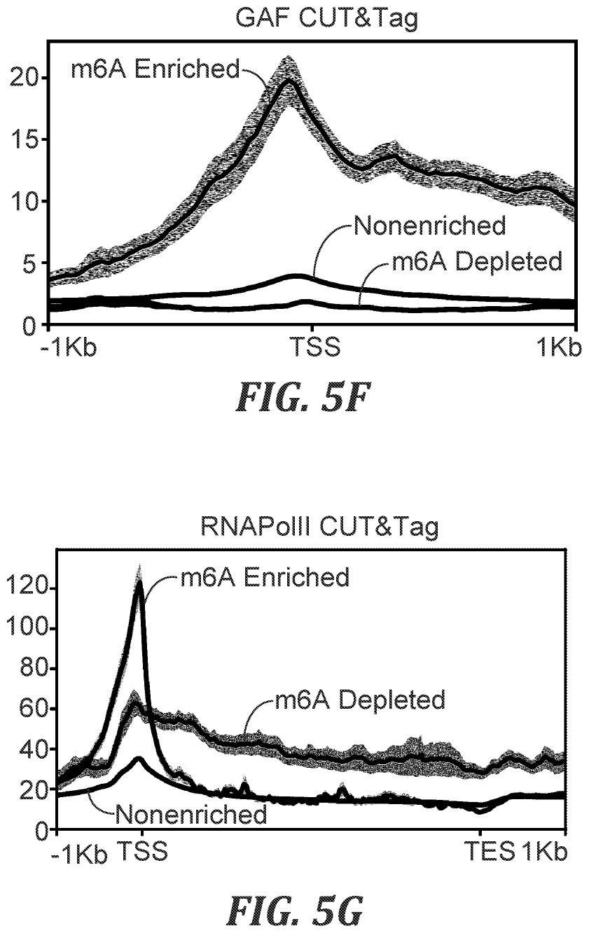

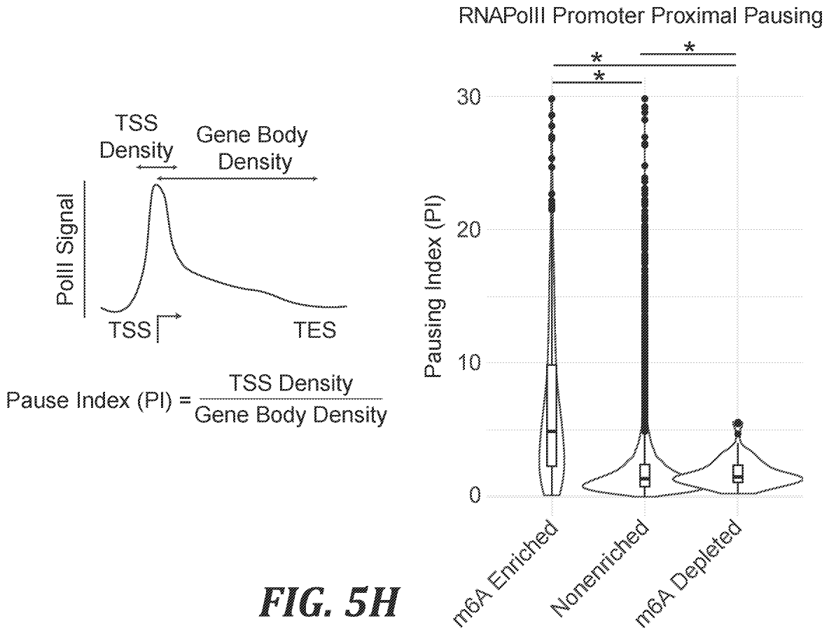

### Genes of Methylated Transcripts are Characterized by Promoter Proximally Paused RNA Polymerase II

Whereas the promoters of genes producing m6A-enriched transcripts are enriched for METTL3, it was noticed that the METTL3 CUT&Tag signal at TSSs of m6A-depleted transcripts was still above IgG CUT&Tag signal (FIG. 14B). In fact, METTL3 binding was widely observed amongst the top 25% expressed genes (>17CPM) (FIG. 5A). Indeed, total RNA polymerase II (RNAPolII) and METTL3 binding are positively correlated (FIG. 5A, FIG. 14C (Haussmann, I. U. et al. m(6)A potentiates Sxl alternative pre-mRNA splicing for robust Drosophila sex determination. Nature 540, 301-304 (2016); Akhtar, J. et al. m(6)A RNA methylation regulates promoter-proximal pausing of RNA polymerase II. Mol Cell 81, 3356-3367 e3356 (2021)). Thus, it was reasoned that METTL3 must be preferentially recruited to sites of active transcription. This leads to the expectation that highly expressed transcripts would be enriched for transcript methylation. However, m6A-enriched transcripts tend to be expressed at lower levels than m6A-depleted transcripts (192 CPM vs 4856 CPM, p=6.2×10⁻⁷; FIG. 5B). In line with expression level differences, genes producing m6A-enriched transcripts have lower levels of active H3K4me3 and H3K36me3 marks (FIG. 5C). Hence, the m6A methylation mark is not associated with high level of transcription. It was then asked whether increasing METTL3 levels at a gene would in turn result in more transcript methylation. Heat shock of Drosophila cells induces a large influx of RNAPolII into the bodies of heat shock protein (HSP) genes (Guertin, M. J., Petesch, S. J., Zobeck, K. L., Min, I. M. & Lis, J. T. Drosophila heat shock system as a general model to investigate transcriptional regulation. Cold Spring Harb Symp Quant Biol 75, 1-9 (2010)), which can be observed by CUT&Tag (FIG. 5D). In addition to RNAPolII enrichment, it was found that heat shock causes a dramatic increase in METTL3 (FIG. 5D). This increase is not limited to promoters, but now extends into the bodies of the Hsp70 genes. However, induced Hsp70 transcripts do not accumulate the m6A modification, despite the large influx of METTL3 and presence of RRACH motifs (the RNA sequence in which the m6A modification occurs) within the Hsp70 transcripts (FIG. 5E, FIG. 14D). Thus, METTL3 binding on its own does not reliably predict methylation status.

What other features might distinguish m6A-enriched and depleted transcripts?Motif analysis revealed GAGA motifs within the promoters of m6A-enriched transcripts (FIG. 14E). GAGA factor (GAF) is a DNA-binding transcription factor that binds GAGA motifs and is associated with promoter proximal pausing of RNAPolII (Chetverina, D., Erokhin, M. & Schedl, P. GAGA factor: a multifunctional pioneering chromatin protein. Cell Mol Life Sci 78, 4125-4141 (2021)). In line with GAGA motif enrichment, much higher GAF CUT&Tag signal is detected at the TSSs of m6A-enriched (FIG. 5F). For this reason, the distribution of total RNAPolII signal over gene bodies relative to the TSS was queried. It was observed m6A-enriched transcripts to have more RNAPolII signal at the TSS and less within gene bodies (FIG. 5G). The RNAPolII promoter proximal Pausing Index (PI) was then calculated as the ratio of RNAPolII signal at the promoter (+250 bp around the TSS) to signal over the gene body. Indeed, m6A-enriched transcripts had very high levels of PI relative to m6A-depleted transcripts (7.4 vs 1.8, p<2.2e-16) (FIG. 5H). This high level of PI was not related to the expression level of the m6A-enriched transcripts (FIG. 14F). Altogether, these findings suggest that transcripts with a very high degree of polymerase pausing and high GAF binding at their promoters are predominantly enriched for the m6A post-transcriptional modification.

In this work RT&Tag was developed, which serves as a proximity labeling tool that uses antibodies to tether Tn5 and tagment nearby RNA within intact nuclei. RT&Tag fundamentally differs from immunoprecipitation-based methods which capture RNA binding to factors within a cell lysate instead of endogenous proximity interactions. Furthermore, RT&Tag does not require cross-linking or RNA fragmentation, and the same RT&Tag protocol can be applied to RNA-protein interactions, RNA-chromatin interactions, and RNA modifications. In contrast, immunoprecipitation techniques require separate protocols for each application.

A major advantage of RT&Tag over immunoprecipitation is its efficiency. RT&Tag requires fewer than ˜100,000 cells which is at least 50-fold fewer than the number needed for PIRCh-seq and ChRIP-seq (Table 1; Fang, J. et al. PIRCh-seq: functional classification of non-coding RNAs associated with distinct histone modifications. Genome Biol 20, 292 (2019); Mondal, T., Subhash, S. & Kanduri, C. Chromatin RNA Immunoprecipitation (ChRIP). Methods Mol Biol 1689, 65-76 (2018)). RT&Tag also works with few sequencing reads as the RT&Tag reads are concentrated at the 3′ end of RNA (Pallares, L. F., Picard, S. & Ayroles, J. F. TM3′seq: A Tagmentation-Mediated 3′ Sequencing Approach for Improving Scalability of RNAseq Experiments. G3 (Bethesda) 10, 143-150 (2020)). Specifically, there was success with 4-8 million reads per sample for RT&Tag, relative to PIRCh-seq where around 50 million reads were used (Table 1; Fang, J. et al. PIRCh-seq: functional classification of non-coding RNAs associated with distinct histone modifications. Genome Biol 20, 292 (2019)). Other enzyme tethering based techniques are emerging as in situ alternatives to immunoprecipitation. For example, APEX sequencing (APEX-seq) and Targets of RNA-binding proteins Identified By Editing (TRIBE) tether RNA modifying enzymes by fusing them with other proteins (Fazal, F. M. et al. Atlas of Subcellular RNA Localization Revealed by APEX-Seq. Cell 178, 473-490 e426 (2019); Padron, A., Iwasaki, S. & Ingolia, N. T. Proximity RNA Labeling by APEX-Seq Reveals the Organization of Translation Initiation Complexes and Repressive RNA Granules. Mol Cell 75, 875-887 e875 (2019); McMahon, A. C. et al. TRIBE: Hijacking an RNA-Editing Enzyme to Identify Cell-Specific Targets of RNA-Binding Proteins. Cell 165, 742-753 (2016)). However, these methods have yet to be used to identify RNA interactions occurring on chromatin. Additionally, the need to generate fusion proteins for each protein target makes these techniques laborious and low throughput, unlike RT&Tag, which can be easily applied to any epitope with an available antibody. Another advantage of RT&Tag is that RNA/cDNA hybrids are directly tagmented by Tn5 with sequencing adaptors. This allows for seamless generation of Illumina sequencing libraries using a simple PCR reaction, without the need to purify RNA as in ChRIP-seq, APEX-seq and TRIBE. The lack of purification steps makes RT&Tag adaptable for automation as was done with AutoCUT&Tag (Janssens, D. H. et al. Automated CUT&Tag profiling of chromatin heterogeneity in mixed-lineage leukemia. Nat Genet 53, 1586-1596 (2021)). Together with low cell number input, low sequencing depth, RT&Tag presents a high throughput method to study RNA metabolism by targeting chromatin factors and post-translational modifications.

TABLE 1
Comparison of RT&Tag to immunoprecipitation-based methods.

| | | | RNA-protein | | | | RNA-chromatin | | | | RNA modification | | | |
|---|---|---|---|---|---|---|---|---|---|---|---|---|---|---|
| | RT&Tag | | RIP-seq | | CLIP-seq | | PIRCh-seq | | ChRIP-seq | | MeRIP-seq | | m6A-RIP-seq | |
| Input | 0.1 | million | 20 | million | 20 | million | 5 | million | 20 | million | 20 | million | 50 | million |
| Material | | | | | | | | | | | | | | |
| Sequencing | 4-8 | million | 20 | million | 25 | million | 50 | million | 50 | million | 73 | million | 20 | million |
| reads | | | | | | | | | | | | | | |
| Time | 1-2 | Days | 3 | Days | 4 | Days | 3 | Days | 3 | Days | 2-3 | Days | 2-3 | Days |

| | | | | | | | |
|---|---|---|---|---|---|---|---|
| Cost | $50 | $150 | $200 | $225 | $200 | $300 | $200 |

Using RT&Tag, insight was gained into the N-methyladenosine (m6A) modification. m6A is the most prevalent mRNA post-transcriptional modification and has been implicated in splicing, mRNA decay, and translation (He, P. C. & He, C. m(6) A RNA methylation: from mechanisms to therapeutic potential. EMBO J40, e105977 (2021)). The m6A modification is catalyzed by the methyltransferase, METTL3 (Lence, T., Soller, M. & Roignant, J. Y. A fly view on the roles and mechanisms of the m(6)A mRNA modification and its players. RNA Biol 14, 1232-1240 (2017)). How METTL3 discriminates which RNAs get methylated is unclear. Widespread METTL3 binding was observed at the promoters of expressed genes. However, it was found that most of these genes were not enriched for m6A, suggesting that other factors must be involved. Instead, RNAPolII promoter pausing was found to be a strong predictor of m6A deposition. It was surprising that Hsp70, a gene known to exhibit RNAPolII pausing, was not identified as being m6A-enriched using RT&Tag. However, upon calculating the pausing index of Hsp70, it was found to be on par with that of m6A nonenriched transcripts. This suggests that only genes exhibiting very high levels of RNAPolII pausing are enriched for m6A. RNAPolII dynamics, especially elongation speed, have previously been implicated in regulating co-transcriptional processes including splicing and alternative polyadenylation (Muniz, L., Nicolas, E. & Trouche, D. RNA polymerase II speed: a key player in controlling and adapting transcriptome composition. EMBO J 40, e105740 (2021)). Furthermore, human MCF7 breast cancer cells expressing a slow elongation RNAPolII mutant have been reported to have increased m6A levels (Slobodin, B. et al. Transcription Impacts the Efficiency of mRNA Translation via Co-transcriptional N6-adenosine Methylation. Cell 169, 326-337 e312 (2017)). How RNAPolII promoter pausing contributes to m6A deposition is not known but can be due to the increased amount of time METTL3 is bound near the promoter. As such, METTL3 would have more contact time with the 5′ end of RNA, the region where m6A is predominantly found in Drosophila (Lence, T. et al. m(6)A modulates neuronal functions and sex determination in Drosophila. Nature 540, 242-247 (2016)). METTL3 itself has been found to promote productive RNAPolII elongation, which suggests that there can be two-way communication between m6A and RNAPolII processivity (Akhtar, J. et al. m(6)A RNA methylation regulates promoter-proximal pausing of RNA polymerase II. Mol Cell 81, 3356-3367 e3356 (2021); Xu, W. et al. Dynamic control of chromatin-associated m(6)A methylation regulates nascent RNA synthesis. Mol Cell (2022)). An alternative explanation for the discrepancy between METTL3 binding and m6A levels is that methylation can occur at all METTL3 bound transcripts but not be retained. Fat mass and obesity-associated protein (FTO) is a demethylase that is known to remove the m6A mark after transcription in mammals. However, no FTO homologue has been identified in Drosophila (Lence, T., Soller, M. & Roignant, J. Y. A fly view on the roles and mechanisms of the m(6)A mRNA modification and its players. RNA Biol 14, 1232-1240 (2017)). Deposition of m6A at splice junctions and introns of nascent transcripts has been implicated in regulating splicing (Louloupi, A., Ntini, E., Conrad, T. & Orom, U. A. V. Transient N-6-Methyladenosine Transcriptome Sequencing Reveals a Regulatory Role of m6A in Splicing Efficiency. Cell Rep 23, 3429-3437 (2018)). Thus, intronic m6A marks can be lost during splicing and not be captured by m6A RT&Tag, which specifically measures m6A levels in mature transcripts. Altogether, these findings suggest METTL3 binding does not correspond to the presence of m6A, and that additional factors are necessary for transcript methylation.

Being a proximity tagmentation tool, RT&Tag can have numerous applications given there is an available antibody. Although this work described only chromatin applications, RT&Tag is not necessarily limited to chromatin, and future studies might adapt RT&Tag for targets in the cytoplasm, such as RNA-protein interactions and RNA post-transcriptional modifications. Efforts to catalogue RNA binding protein (RBP) bound transcripts are still in their infancy. Phase 3 of the ENCODE consortium profiled 150 RBPs using immunoprecipitation in the HepG2 and K562 cell lines (Van Nostrand, E. L. et al. A large-scale binding and functional map of human RNA-binding proteins. Nature 583, 711-719 (2020)). Given that the human genome contains over 1500 RBP-encoding genes and mutations in RBPs are becoming implicated in genetic diseases, much work remains to be done to characterize their bound transcripts (Gerstberger, S., Hafner, M. & Tuschl, T. A census of human RNA-binding proteins. Nat Rev Genet 15, 829-845 (2014); Gebauer, F., Schwarzl, T., Valcarcel, J. & Hentze, M. W. RNA-binding proteins in human genetic disease. Nat Rev Genet 22, 185-198 (2021)). Similarly, cataloguing sites of m6A modification on a large scale is yet to be done. METTL3 knock-out experiments in mammals (human and mice) have shown that m6A is required for cell differentiation and embryonic viability (Geula, S. et al. Stem cells. m6A mRNA methylation facilitates resolution of naive pluripotency toward differentiation. Science 347, 1002-1006 (2015); Li, H. B. et al. m(6)A mRNA methylation controls T cell homeostasis by targeting the IL-7/STAT5/SOCS pathways. Nature 548, 338-342 (2017); Lee, H. et al. Stage-specific requirement for Mettl3-dependent m(6)A mRNA methylation during haematopoietic stem cell differentiation. Nat Cell Biol 21, 700-709 (2019); Batista, P. J. et al. m(6)A RNA modification controls cell fate transition in mammalian embryonic stem cells. Cell Stem Cell 15, 707-719 (2014)). The commonly used MeRIP-seq and m6A-seq techniques require large amounts of RNA input which makes them impractical for studying differentiating cells and development. RT&Tag can fill the need for high throughput profiling of chromatin-bound RBP-RNA interactions and m6A enriched transcripts, especially when sample input is limiting such as with clinical samples or embryonic cells.

### Cell Culture and Nuclei Preparation

Drosophila S2 cells were obtained from Invitrogen (10831-014) and were cultured in HyClone SFX-Insect cell culture media (HyClone) supplemented with 18 mM L-Glutamine (Sigma-Aldrich). S2 cells were maintained at the confluency of 2-10 million cells per mL at 25° C. To induce the heat shock response, S2 cells were placed at 37° C. for 15 minutes. To prepare nuclei for CUT&Tag and RT&Tag, 4 million S2 cells were collected by centrifuging at 300 g for 5 minutes followed by a wash with 1×PBS. Nuclei were then isolated by incubating with NE1 buffer (10 mM HEPES pH7.9, 10 mM KCl, 0.1% Triton X-100, 20% glycerol, 0.5 mM spermidine, Roche Complete Protease Inhibitor Cocktail) for 10 minutes on ice. The nuclei were then centrifuged at 500 g for 8 minutes and resuspended in Wash Buffer (20 mM HEPES pH7.5, 150 mM NaCl, 0.5 mM spermidine, Roche Complete Protease Inhibitor Cocktail). The nuclei were either used fresh or were frozen in Wash Buffer with 10% DMSO and stored at −80° C. For RT&Tag, the NE1 and Wash buffers were supplemented with 1 U/μL of RNasin Ribonuclease Inhibitor (Promega).

### Antibodies

The following primary antibodies were used for RT&Tag and CUT&Tag experiments: rabbit anti-IgG (Abcam ab172730), rabbit anti-MSL2 (gift from Mitzi Kuroda, Harvard Medical School), rabbit anti-H4K16ac (Abcam ab109463), rabbit anti-H3K27me3 (Cell Signaling Technology CST9733), rabbit anti-H3K36me3 (Thermo MA5-24687), rabbit anti-H3K4me3 (Thermo 711958), rabbit anti-m6A (Megabase AP60500), rabbit anti-METTL3 (Proteintech 15073-1-AP), mouse anti-unphosphorylated RNA polymerase II (Abcam ab817), rabbit anti-GAF (gift from Giovanni Cavalli, CNRS Montpellier France). The following secondary antibodies were used: Guinea Pig anti-Rabbit (Antibodies Online ABIN101961) and Rabbit anti-Mouse (Abcam ab46450). Streptavidin conjugated secondary antibodies were generated using the Streptavidin Conjugation Kit (Abcam ab102921) as per manufacturer's instructions.

### RT&Tag

Single loaded pA-Tn5 was assembled prior to starting RT&Tag. First, the Mosaic end-adapter A (ME-A) and its reverse (ME-Rev) oligonucleotides were annealed in Annealing Buffer (10 mM Tris pH8, 50 mM NaCl, 1 mM EDTA) by heating them at 95° C. for 5 minutes and slowly allowing them to cool to room temperature. Afterwards, 16 μL of 100 μM annealed ME-A were mixed with 100 μL of 5.5 μM pA-Tn5 for 1 hour at room temperature and stored at −20° C. for future use. S2 nuclei were isolated and bound to paramagnetic Concanavalin A (ConA) beads (Bangs Laboratories). To do so, ConA beads were first activated via 2 washes with Binding Buffer (10 mM HEPES pH7.9, 10 mM KCl, 1 mM CaCl₂), 1 mM MnCl₂). Afterwards, 100,000 S2 nuclei were bound to 5 μL of ConA beads for 10 minutes at room temperature. The ConA bound nuclei were then incubated with primary antibody diluted 1:100 in Antibody Buffer (20 mM HEPES pH7.5, 150 mM NaCl, 0.5 mM spermidine, Roche Complete Protease Inhibitor Cocktail, 2 mM EDTA, 0.1% BSA and 1 U/μL RNasin Ribonuclease Inhibitor) at 4° C. overnight. Afterwards, nuclei were incubated with streptavidin conjugated secondary antibody diluted 1:100 in Wash Buffer (20 mM HEPES pH7.5, 150 mM NaCl, 0.5 mM spermidine, Roche Complete Protease Inhibitor Cocktail) for 45 minutes at RT. Two rounds of washes with Wash Buffer were then performed and nuclei were incubated with 0.2 mM biotinylated oligo(dT)-ME-B in Wash Buffer for 20 minutes at RT. Two rounds of washes with Wash Buffer were then performed and nuclei were incubated with ME-A loaded pA-Tn5 diluted 1:200 in 300 Wash Buffer (20 mM HEPES pH7.5, 300 mM NaCl, 0.5 mM spermidine, Roche Complete Protease Inhibitor Cocktail, and 1 U/μL RNasin Ribonuclease Inhibitor) for 1 hour at RT. ConA bound nuclei were then washed thrice with 300 Wash Buffer. Simultaneous reverse transcription and tagmentation were then performed by resuspending nuclei in MgCl₂ containing Reverse Transcription buffer (1× Maxima RT Buffer which contains 50 mM Tris-HCl pH 8.3, 75 mM KCl, 3 mM MgCl₂, 10 mM DTT along with, 0.5 mM dNTPs, 10 U/μL of Maxima H Minus Reverse Transcriptase, and 1 U/μL of RNasin Ribonuclease Inhibitor) for 2 hours at 37° C. The nuclei were then washed with 10 mM TAPS and pA-Tn5 was stripped off by resuspending nuclei in 5 μL of Stripping Buffer (10 mM TAPS with 0.1% SDS) and incubating for 1 hour at 58° C. Libraries were then generated using PCR. The nuclei suspension was mixed with 15 μL of 0.67% Triton X-100, 2 μL of 10 mM i7 primer, 2 μL of 10 mM i5 primer and 25 μL of 2×NEBNext Master Mix (NEB). The following PCR conditions were used: 1) 58° C. for 5 minutes, 2) 72° C. for 5 minutes, 3) 98° C. for 30 seconds, 4) 98° C. for 10 seconds, 5) 60° C. for 15 seconds, 6) Repeat steps 4-5 13 times, 7) 72′C for 2 minutes, 8) Hold at 4° C. Sequencing libraries were then purified using 0.8× HighPrep PCR Cleanup System (MagBio) beads as per manufacturer's instructions. Libraries were then resuspended in 21 μL of 10 mM Tris-HCl pH8. Library concentrations were quantified using the High Sensitivity D5000 TapeStation system (Agilent).

### CUT&Tag

CUT&Tag was carried out as described prior (Protocols website Cut&Tag-direct with CUTAC V.3) (WO2019060907; Kaya-Okur, H. S. et al. CUT&Tag for efficient epigenomic profiling of small samples and single cells. Nat Commun 10, 1930 (2019), each of which is incorporated herein by reference in its entirety). Briefly, S2 nuclei were bound to ConA beads at the ratio of 100,000 nuclei per 5 μL beads for 10 minutes at room temperature. Nuclei were then incubated with primary antibody (1:100) at 4° C. overnight followed by secondary antibody (1:100) for 45 minutes at RT the next day. Excess antibody was removed via 2 rounds of washes and the nuclei were incubated with loaded pA-Tn5 (1:200) for 1 hour at RT. Nuclei were washed thrice to remove excess pA-Tn5 and then MgCl₂ was added to perform tagmentation for 1 hour at 37° C. The reaction was then stopped by doing a wash with 10 mM TAPS and stripping off pA-Tn5 by resuspending nuclei in 0.1% SDS buffer and incubating for 1 hour at 58° C. The SDS was then neutralized with Triton X-100 and libraries were amplified with NEBNext Master Mix (NEB) using 12 rounds of amplification. Sequencing libraries were then purified using 1.2× ratio of HighPrep PCR Cleanup System (MagBio) as per manufacturer's instructions. Libraries were then resuspended in 21 μL of 10 mM Tris-HCl pH8. Library concentrations were quantified using the D1000 TapeStation system (Agilent).

### RNA Interference (RNAi)

PCR templates for in vitro transcription (IVT) were amplified from S2 cell cDNA or pGFP5(S65T) plasmid using Phusion Hot Start Flex DNA Polymerase (NEB) and primers listed in Supplementary Table 2. PCR products were purified using NucleoSpin® Gel and PCR Clean-Up Kit (Clontech). IVT was performed to generate dsRNA using the T7 High Yield RNA Synthesis Kit (NEB). Template DNA was removed using Turbo DNAse (Ambion) and dsRNA was purified using the NucleoSpin® RNA Clean-up XS kit (Clontech). To perform RNAi, S2 cells were seeded at a density of 1 million cells/mL of serum-free medium. As control RNAi, a total of 30 μg of GFP dsRNA was added to cells. For Mettl3 RNAi, 15 μg of Mettl3 dsRNA #1 plus 15 μg of Mettl3 dsRNA #2 were added. After 6 hours, medium was replaced with serum containing medium. Treatment with dsRNA was repeated after 48 and 96 hours. Cells were collected after 120 hours.

### RT-qPCR

Total RNA was extracted from S2 cells using the RNeasy Plus Mini Kit (Qiagen) according to manufacturer's instructions. cDNA was synthesized using the Maxima H Minus Reverse Transcriptase (Thermo Scientific). Real time PCR was performed with the Maxima™ SYBR™ Green qPCR Master Mix (Thermo Scientific) using the ABI QuantStudio5 Real Time PCR Systems instrument. Primers used are listed in Supplementary Table 3. Gene expression levels were quantified using the delta delta Ct method using the Ribosomal Protein L32 (RPL32) gene for normalization.

### RNA-Sequencing

Total RNA from S2 cells was isolated using the RNeasy Plus Mini Kit (Qiagen). Maxima H Minus Reverse Transcriptase (Thermo Fisher Scientific) was used as per manufacturer's instructions for first strand synthesis. Reverse transcription was primed using the oligo(dT)-ME-B fusion oligonucleotide. Tagmentation was then performed using 100ng of RNA-cDNA hybrids, ME-A loaded pA-Tn5, and tagmentation buffer (20 mM HEPES pH7.5, 150 mM NaCl, 10 mM MgCl₂) for 1 hour at 37° C. Tagmented RNA-cDNA hybrids were purified using 1× ratio of HighPrep PCR Cleanup System (MagBio) as per manufacturer's instructions. Sequencing libraries were then amplified using NEBNext Master Mix (NEB) using 12 cycles. Libraries were then purified using 0.8× ratio of HighPrep PCR Cleanup System (MagBio) as per manufacturer's instructions. Libraries were then resuspended in 21 μL of 10 mM Tris-HCl pH8 and quantified using the D5000 TapeStation system (Agilent).

### Sequencing and Data Preprocessing

For RT&Tag and RNA-sequencing, single-end 50 base pair sequencing was performed on the Illumina HiSeq. The sequencing reads were aligned using HISAT2 to the UCSC dm6 genome with the options: --max-intronlen 5000--ma-strandness F (Kim, D., Paggi, J. M., Park, C., Bennett, C. & Salzberg, S. L. Graph-based genome alignment and genotyping with HISAT2 and HISAT-genotype. Nat Biotechnol 37, 907-915 (2019)). The aligned reads were then quantified using featureCounts with the Ensembl dm6 gene annotation file using the following options: -s 1-t exon -g gene_id (Liao, Y., Smyth, G. K. & Shi, W. featureCounts: an efficient general purpose program for assigning sequence reads to genomic features. Bioinformatics 30, 923-930 (2014)). HISAT2 alignment statistics, PCR duplication rate (Samtools markdup (Li, H. et al. Differential expression and principal component analysis were performed using DESeq2 (Love, M. I., Huber, W. & Anders, S. Moderated estimation of fold change and dispersion for RNA-seq data with DESeq2. Genome Biol 15, 550 (2014)). The genomic origin of RT&Tag reads was determined using QualiMap RNA-Seq QC (Okonechnikov, K., Conesa, A. & Garcia-Alcalde, F. Qualimap 2: advanced multi-sample quality control for high-throughput sequencing data. Bioinformatics 32, 292-294 (2016)). IgG normalized MSL2 RT&Tag signal was visualized over the Drosophila chromosomes using karyotypeR (Gel, B. & Serra, E. karyoploteR: an R/Bioconductor package to plot customizable genomes displaying arbitrary data. Bioinformatics 33, 3088-3090 (2017)). GO term enrichment analysis for H3K27me3 and m6A enriched or depleted transcripts was performed using clusterProfiler (Yu, G., Wang, L. G., Han, Y. & He, Q. Y. clusterProfiler: an R package for comparing biological themes among gene clusters. OMICS 16, 284-287 (2012)). The distribution of RT&Tag reads across the gene bodies of drosophila genes was calculated using RSeQC (Wang, L., Wang, S. & Li, W. RSeQC: quality control of RNA-seq experiments. Bioinformatics 28, 2184-2185 (2012)). For CUT&Tag, paired-end 25 base pair sequencing was performed on the Illumina HiSeq and data was analyzed as described prior (dx.doi.org/10.17504/protocols.io.bjk2kkye) (WO2019060907; Kaya-Okur, H. S. et al. CUT&Tag for efficient epigenomic profiling of small samples and single cells. Nat Commun 10, 1930 (2019)). MSL2 and H3K27me3 peaks were called using SEACR using the norm setting (Meers, M. P., Tenenbaum, D. & Henikoff, S. Peak calling by Sparse Enrichment Analysis for CUT&RUN chromatin profiling. Epigenetics Chromatin 12, 42 (2019)). Profile plots, heatmaps and correlation matrices were generated using deepTools (Ramirez, F. et al. deepTools2: a next generation web server for deep-sequencing data analysis. Nucleic Acids Res 44, W160-165 (2016)). RRACH motifs were identified using the FIMO tool from the MEME suit (Grant, C. E., Bailey, T. L. & Noble, W. S. FIMO: scanning for occurrences of a given motif. Bioinformatics 27, 1017-1018 (2011)). Motif enrichment within the promoters of m6A enriched vs depleted transcripts was performed using the MEME tool from the MEME suit using the differential enrichment mode (Bailey, T. L. & Elkan, C. Fitting a mixture model by expectation maximization to discover motifs in biopolymers. Proc Int Conf Intell Systi Mol Biol 2, 28-36 (1994)). Genome browser screenshots were obtained from the University of California Santa Cruz (UCSC) Genome Browser. Graphs were plotted using R Studio (r-project.org) using base graphics or using ggplot2 (ggplot2 website).

### Example 2

This Example discloses the use of RT&Tag to profile chromatin-associated RNAs in mammalian cells.

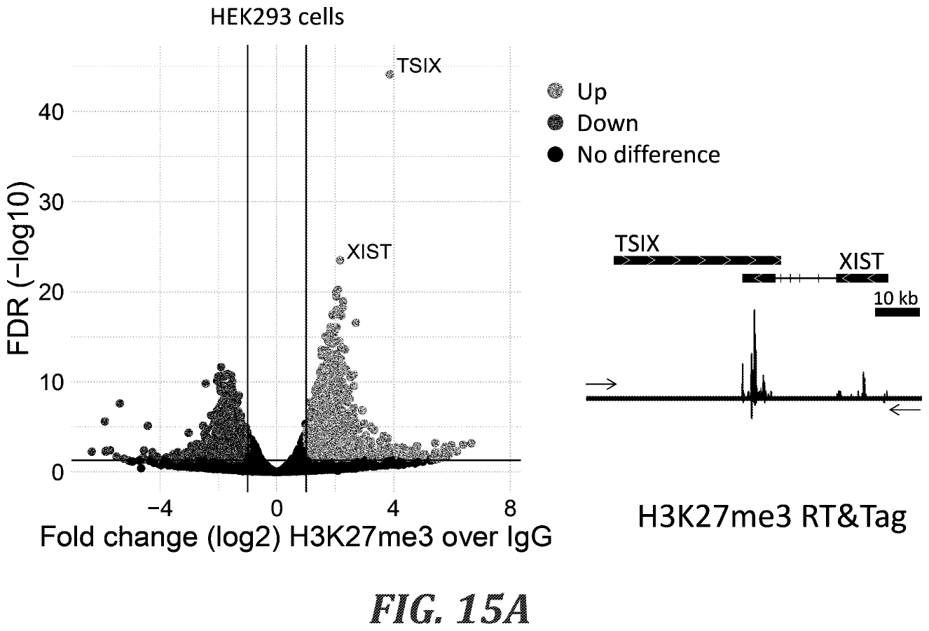

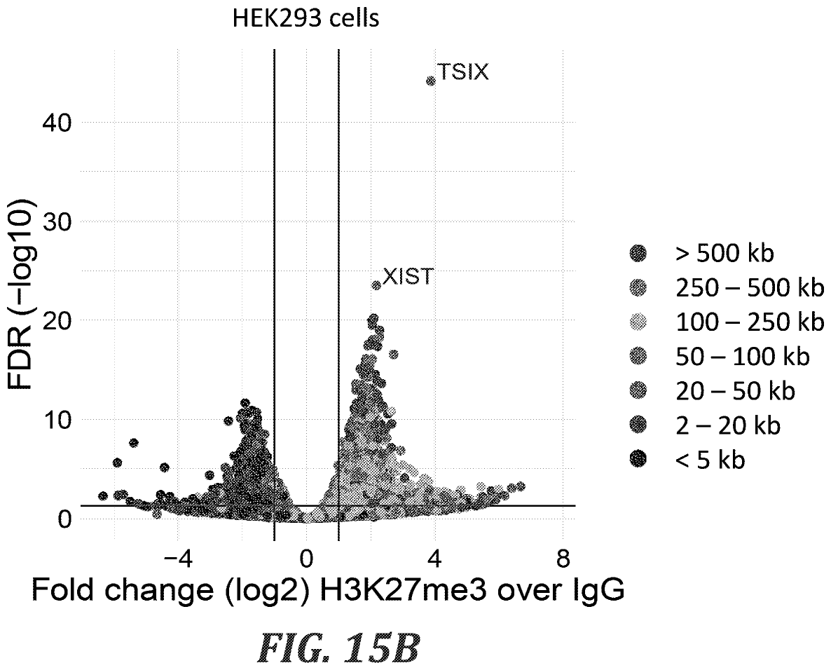

RT&Tag was developed and tested in Drosophila cells where the small genome sped analysis. RT&Tag has been tested with human cell lines and the protocol works efficiently. Given the wealth of literature implicating RNA in targeting Polycomb silencing to the inactivated X chromosome in mammalian female cells, RT&Tag was performed using antibodies targeting the H3K27me3 mark (or IgG as background control) with 200,000 female K562 cells. DESeq2 was used to call transcripts that were differentially enriched for H3K27me3 over IgG (fold change >2, FDR <0.05). These experiments recovered the 3′-overlapping IncRNAs, Xist, and its repressor Tsix, which are known to be localized to the inactive and active X chromosomes respectively (FIGS. 15A and 15B), as well as rare transcripts from Polycomb-silenced domains. This demonstrates that RT&Tag has the sensitivity to detect regulatory chromatin-associated RNAs.

RT&Tag will be applied to an in vitro differentiation time-course of female mouse embryonic stem cells (mESCs) to follow changes in the Xist IncRNA and the progression of silencing of new mRNA production from the X chromosome. Mouse cells will be used because culture conditions for X chromosome inactivation from pluripotent cells has been established, while X inactivation in human cell culture models are incomplete. In female mESCs both X chromosomes are active, and inactivation then initiates upon in vitro differentiation. One of the first events is transcription of the Xist gene from the inactivating chromosome, and these transcripts go on to coat the chromosome. Successive reduction of RNA polymerase and the establishment of repressive histone modifications across the chromosome follow Payer, B et al., (Payer B, Lee JT. X chromosome dosage compensation: how mammals keep the balance. Annu Rev Genet. 2008; 42:733-72). To track the production and localization of Xist RNA, an antibody to its binding protein SAF-A (Hasegawa Y, et al., The matrix protein hnRNP U is required for chromosomal localization of Xist RNA. Dev Cell. 2010 Sep. 14; 19(3):469-76) will be used for RT&Tag. These experiments will track the localization of the Xist-SAF-A complex to the inactivating chromosome through detection of X-linked transcripts, as observed in Drosophila cells. See e.g., Example 1. Parallel experiments will profile the onset of repressive histone modifications by CUT&Tag, connecting the establishment and spread of repressive marks with the timeline of Xist chromatin localization.

### Example 3

This Example discloses the use of RT&Tag to annotate the half-lives of chromatin-associated RNAs by SLAM-RT&Tag.

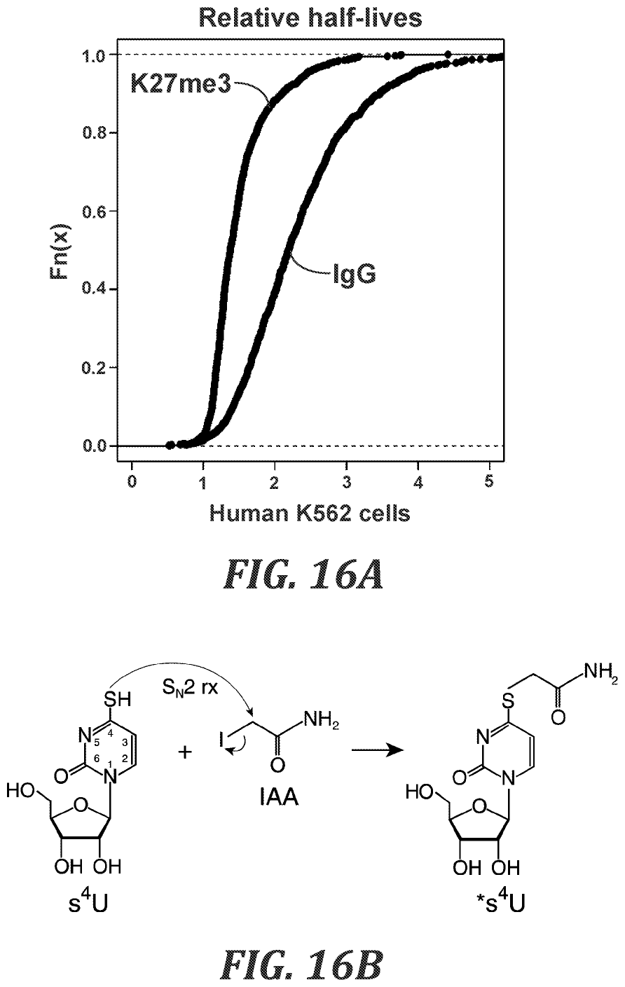

Candidate IncRNAs can be identified by transcriptomic studies, but implicating functions require more time-consuming experiments. However, the stability of many IncRNAs is critical for their function, and thus metabolic labeling offers a high-throughput method to distinguish potential functions of any chromatin-associated RNAs. To measure the half-lives of individual chromatin-associated RNAs, various RNA labeling moieties and chemistries have been tried, the SLAM ((SH)-Linked Alkylation for the Metabolic sequencing of RNA) method of metabolic RNA labeling (Herzog V A et al., Thiol-linked alkylation of RNA to assess expression dynamics. Nat Methods. 2017 December; 14(12):1198-1204) was successfully combined with RT&Tag. K562 cells were pulse-labeled with 100 mM 4-thiouridine (4sU) for 4 hours and then chased for various times in fresh media with excess uridine. Isolated nuclei were bound to concanavalin A magnetic beads and treated with iodoacetamide to alkylate labeled RNA to cytosine before proceeding with H3K27me3-tethered RT&Tag. For analysis, the SLAM-DUNK tool (Neumann T, et al., Quantification of experimentally induced nucleotide conversions in high-throughput sequencing datasets. BMC Bioinformatics. 2019 May 20; 20(1):258) was used to count labeled transcripts and infer their half-lives in chases. With ˜5% labeling efficiency, there are sufficient counts of labeled and unlabeled RT&Tag products to estimate relative half-lives for RNAs from Polycomb-silenced domains (FIG. 16). These experiments reveal that transcripts from Polycomb-silenced domains turn-over unusually fast, consistent with the idea that these are aberrant non-productive transcripts. In contrast, the Xist IncRNA is more stable than bulk RNA, consistent with its role in coating the inactive X chromosome.

Based on the results from the turn-over experiments described above, 5′RT&Tag will be applied to the developmental germ layer time-courses described in Example 2. Half-lives should be informative for distinguishing potentially functional RNAs from aberrant transcripts; for example, functional IncRNAs should be stable, like roX2 and Xist IncRNAs. Recent work has implicated Polycomb-mediated targeting of RNA processing enzymes in the degradation of nascent transcripts from repressed genes (Zhou H, et al., Rixosomal RNA degradation contributes to silencing of Polycomb target genes. Nature. 2022 April; 604(7904):167-174), and this parallels the observations of aberrant transcripts from Hox genes in Drosophila and in human cells. The relative timing of transcriptional silencing and RNA processing defects will be determined in the tri-lineage time-courses where different developmental genes are gaining or losing Polycomb-silencing. To directly assess potential defects in transcript processing, antibodies to splicing proteins will be used to measure the intermediate states as splicing proceeds. The inventors have a particular interest in the splicing factor SF3B1, which interacts both with nucleosomes and with Polycomb proteins (Isono K, et al., Mammalian polycombmediated repression of Hox genes requires the essential spliceosomal protein Sf3bl. Genes Dev. 2005 Mar. 1; 19(5):536-41; Kfir N, et al., SF3B1 association with chromatin determines splicing outcomes. Cell Rep. 2015 April28; 11(4):618-29), whose binding to chromatin will be analyze by CUT&Tag and binding to RNA by RT&Tag through tethering to SF3B or other spliceosome components. The significance of enhancing RT&Tag so that nascent RNA half-lives are determined (SLAM-RT&Tag) can further distinguish potential regulatory RNA function from the background of transient nascent transcription.

### Example 4

This Example discloses examples for expanding the capabilities of RT&Tag.

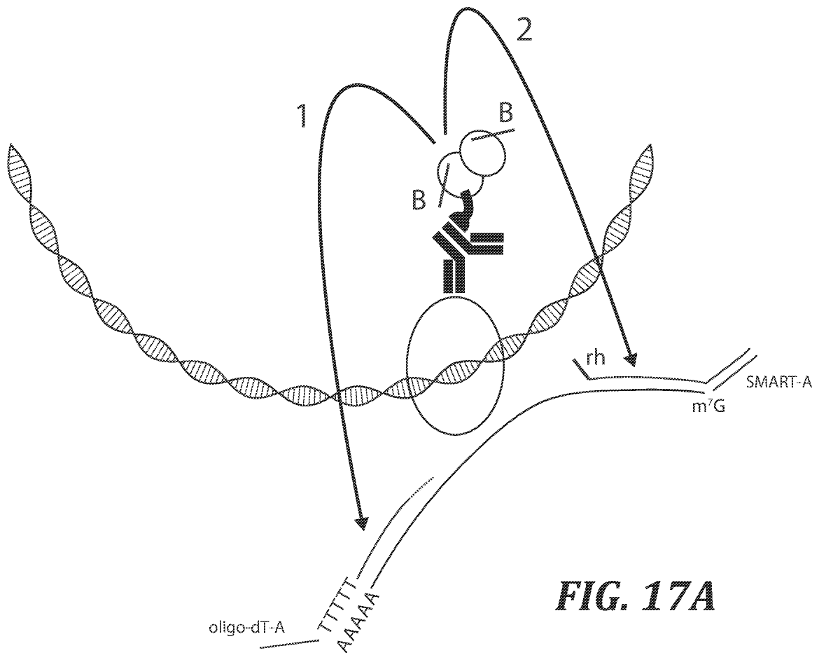

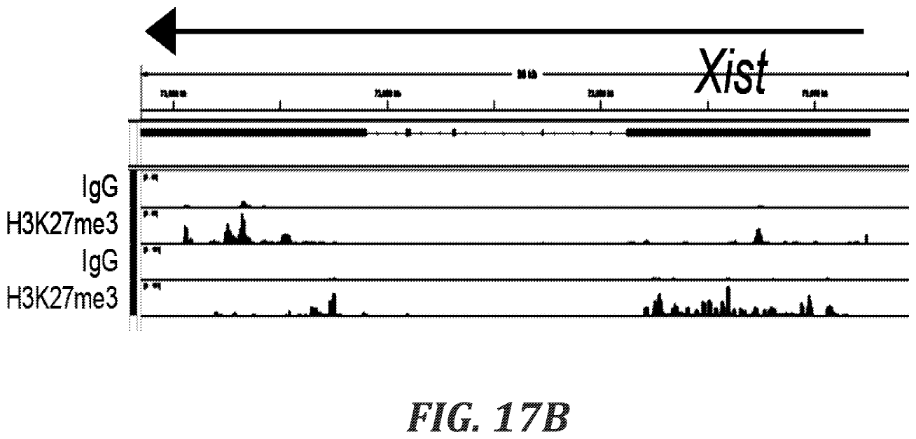

As described in Example 1, RT&Tag uses an oligo-dT-adapter for priming reverse transcription and is thus compatible with complete mRNA transcripts. It is desirable to capture both incomplete mRNA transcripts as well as other RNA species that lack polyadenylation. For example, despite using an oligo-dT primer, in some cases, the inventors have detected apparent nascent transcripts where RT priming occurs at internal oligo(A) tracts, leading to RT&Tag signal distributed across the bodies and introns of genes. To develop reagents to efficiently capture such transcripts, the inventors have modified the components needed for RT&Tag. In preliminary work, one successful approach has been to use random hexamers to prime reverse transcription, and a biotinylated adapter for template switching of reverse transcriptase once it reaches the 5′ end of transcripts (FIG. 17A). Such SMART adapters have been used in transcriptomic strategies to capture the 5′ end of transcripts (Pintacuda G, Cerase A. X Inactivation Lessons from Differentiating Mouse Embryonic Stem Cells. Stem Cell Rev Rep. 2015 October; 11(5):699-705); thus, the inventors have termed this variation 5′RT&Tag. 5′RT&Tag follows the standard RT&Tag workflow, processing, and computational analysis. As proof-of-principle, H3K27me3-tethered 5′RT&Tag was performed using 200,000 K562 nuclei. Inspection of mapped recovered sequences shows that while RT&Tag reads are concentrated at the 3′ end of the Xist RNA, 5′RT&Tag reads are distributed throughout the two exons of this IncRNA.

This development enables querying other kinds of chromatin-associated RNAs by RT&Tag. For example, a substantial fraction of chromatin-associated RNAs are nascent transcripts anchored to chromatin through engaged RNA polymerases, and these may regulate the activity of chromatin-modifying enzymes (including EZH2 and chromatin remodelers). Additionally, some mRNA lack poly(A) tails, such as the replication-dependent histone gene transcripts. The 5′RT&Tag method will be applied to mESCs differentiated in culture to complement our analyses Example 2. Further, this method allows for efficient detection of intronic sequences of preprocessed mRNA, providing a measure of the efficiency of transcript splicing.

Further, extending RT&Tag to the full mRNA with 5′RT&Tag and not just 3′ ends is significant for probing the RNA interactome with promoter and gene body chromatin components, filling in the gap in the central dogma between transcription by RNA Polymerase and RNA processing that begins on nascent transcripts and continues through export to the cytoplasm. Moreover, 5′RT&Tag can potentially be modified for the study of structural RNAs that are thought to have regulatory functions, such as tRNAs and other RNA Polymerase III transcripts (e.g. Liu X, et al., A prometastatic tRNA fragment drives Nucleolin oligomerization and stabilization of its bound metabolic mRNAs. Mol Cell. 2022 Jul. 21; 82(14):2604-2617).

TABLE 2
FIG. 7 identification

| | | SEQ ID |
|---|---|---|
| Description | Sequence | NO: |
| Tn5-ME-A | TCGTCGGCAGCGTCAGATGTGTATAAGAGACAG | 4 |
| (left side upper | | |
| strand) | | |
| Tn5-ME-A | CTGTCTCTTATACACATCT | 5 |
| (left side lower | | |
| strand) | | |
| Tn5-ME-A | GACAGAGAATATGTGTAGACTGCGACGGCTGCT | 6 |
| (right side | | |
| upper strand) | | |
| Tn5-ME-A | TCTACACATATTCTCTGTC | 7 |
| (right side | | |
| lower strand) | | |
| oligo(dT)-ME- | GTCTCGTGGGCTCGGAGATGTGTATAAGAGACAGTTTT | 8 |
| B | TTTTTTTTTTTTTTTTTTTTTTTTTTTVN | |
| Poly-A tail | AAAAAAAAAAAAAAAAAAAAAAAAA | 9 |
| 15 ME | TCGTCGGCAGCGTCAGATGTGTATAAGAGACAG | 10 |
| ME | CTGTCTCTTATACACATCT | 11 |
| i5 Primer | AATGATACGGCGACCACCGAGATCTACACNNNNNNNN | 12 |
| | TCGTCGGCAGCGTC | |
| i7 Primer | GGCTCGGGTGCTCTGNNNNNNNNTAGAGCATACGGCA | 13 |
| | GAAGACGAAC | |
| Read 1 (top | AATGATACGGCGACCACCGAGATCTACACNNNNNNNN | 14 |
| sequence) | TCGTCGGCAGCGTCAGATGTGTATAAGAGACAG | |
| Read 1 (bottom | CTGTCTCTTATACACATCTGACGCTGCCGACGANNNNN | 15 |
| sequence) | NNNGTGTAGATCTCGGTGGTCGCCGTATCATT | |
| Index 1 (top | AAAAAAAAAAAAAAAAAAAAAAAAAAAACTGTCTCTT | 16 |
| sequence) | ATACACATCTCCGAGCCCACGAGACNNNNNNNNATCT | |
| | CGTATGCCGTCTTCTGCTTG | |
| Index 1 | CAAGCAGAAGACGGCATACGAGATNNNNNNNNGTCTC | 17 |
| (bottom | GTGGGCTCGGAGATGTGTATAAGAGACAGTTTTTTTTT | |
| sequence) | TTTTTTTTTTTTTTTTTTTTTVN | |

TABLE 3
RNAi sequence identification

| | | | SEQ |
|---|---|---|---|
| | | | ID |
| Description | Direction | Sequence | NO: |
| GFP | F | taatacgactcactatagg | 19 |
| (control) | | gagaCCACGATGGCGATGT | |
| RNAi | | TAATGGG | |
| GFP | R | taatacgactcactatagg | 20 |
| (control) | | gagaCCACAGGATCATGTG | |
| RNAi | | ATCTC | |
| Mett13 | F | ttaatacgactcactatag | 21 |
| RNAi | | ggagaCAGCCTGGAGATGG | |
| #1 | | TGAACT | |
| Mett13 | R | ttaatacgactcactatag | 22 |
| RNAi | | ggagaTCAGGCACTCGACT | |
| #1 | | TTTGTG | |
| Mett13 | F | ttaatacgactcactatag | 23 |
| RNAi | | ggagaGCACTGGTCATTGG | |
| #2 | | CTAAAC | |
| Mett13 | F | ttaatacgactcactatag | 24 |
| RNAi | | ggagaCATCTGGATAACGC | |
| #2 | | TTCTGGA | |

TABLE 4
Gene sequence identification

| Gene | Gene ID/ | | SEQ ID |
|---|---|---|---|
| name | Direction | Sequence | NO: |
| RPL32 | NM_170461/ | AGCATACAGGCCCAAGATCG | 25 |
| | F | | |
| RPL32 | NM_170461/ | TGTTGTCGATACCCTTGGGC | 26 |
| | R | | |
| Mett13 | NM_142947/ | AAGGAACTCGTTGAGGCTGA | 27 |
| | F | | |
| Mett13 | NM_142947/ | CACCTGTGTGGAGACAATGG | 28 |
| | R | | |

While illustrative embodiments have been illustrated and described, it will be appreciated that various changes can be made therein without departing from the spirit and scope of the invention.

1. An in-situ method for mapping a cellular RNA or its associated proteins using a tethered enzyme complex, the method comprising the steps of:
a) binding a nucleus, an organelle, a cell, or a tissue to a solid support;
b) permeabilizing the nucleus, the organelle, the cell or the tissue;
c) binding a first recognition agent that binds an epitope of the RNA or its associated proteins;
d) binding a second recognition agent that specifically binds to the first recognition agent, wherein the second recognition agent is conjugated to a biotin-binding moiety;
e) tethering at least one molecule required for reverse transcription comprising a biotinylated oligonucleotide for cDNA synthesis priming and for PCR amplification to the second recognition agent;
f) tethering a transposase fused to protein A (pA-transposase) comprising a first sequencing adapter sequence to the first recognition agent and the second recognition agent;
g) allowing the at least one molecule required for reverse transcription to convert a mature transcript near the binding site of the first recognition agent to an RNA/DNA hybrid comprising a first sequencing adapter sequence and a priming sequence;
h) allowing the pA-transposase to tagment the RNA/DNA hybrid; and
i) preparing sequencing libraries of the RNA/DNA hybrid.

2. The method of claim 1, wherein the at least one molecule required for reverse transcription comprises a first sequencing adapter sequence and a priming sequence, and a biotinylated reverse transcriptase.

3. The method of claim 2, wherein the reverse transcriptase adds three non-templated deoxycytidines (+CCC) to the 3′ end of a cDNA strand, which is then hybridized with an oligonucleotide comprising GGG nucleotides and a sequencing adapter for template-switching extension by the reverse transcriptase.

4. The method of claim 1, wherein both the biotinylated priming sequence, comprising a second sequencing adapter, and the biotinylated reverse transcriptase are tethered to a streptavidin-conjugated second recognition agent by a biotin-streptavidin interaction.

5. The method of claim 1, wherein the at least one molecule required for reverse transcription comprises a biotinylated priming sequence comprising a second sequencing adapter.

6. The method of claim 1, wherein the biotinylated priming sequence is tethered to the streptavidin conjugated second recognition agent by a biotin-streptavidin interaction.

7. The method of claim 1, wherein the pA-transposase is tethered to the first recognition agent and the second recognition agent by Protein A in the pA-transposase binding to the first recognition agent and by Protein A in the pA-transposase binding to the second recognition agent.

8. The method of claim 1, wherein the first recognition agent is a first antibody that specifically binds to an epitope of the RNA.

9. The method of claim 1, wherein the second recognition agent is a second antibody that specifically binds to the first antibody.

10. The method of claim 1, wherein reverse transcription and tagmentation are performed simultaneously.

11. The method of claim 1, wherein reverse transcription is completed before starting tagmentation.

12. The method of claim 1, wherein the second sequencing adapter comprises an i7 adapter, optionally wherein the i7 adapter is appended 5′ to the biotinylated priming sequence.

13. (canceled)

14. The method of claim 12, wherein the sequencing libraries comprise RNA, optionally signal from the 3′ end of RNA.

15. (canceled)

16. The method of claim 1, wherein the first sequencing adapter comprises an i5 adapter.

17. The method of claim 1, wherein the solid support comprises a bead.

18. The method of claim 1, wherein the first sequencing adapter or the second sequencing adapter further comprise a barcode sequence.

19. The method of claim 1, wherein the transposase comprises a Tn5 transposase.

20. The method of claim 1, wherein the pA-transposase comprises a pA-Tn5 transposase.

21. The method of claim 1, wherein the nucleus, the organelle, the cell, or the tissue are isolated from a eukaryotic sample, optionally a human sample.

22. (canceled)

23. The method of claim 1, wherein the epitope of the RNA identifies an RNA-protein interaction, an RNA-chromatin interaction, or an RNA post-transcriptional modification.

24-25. (canceled)

26. The method of claim 3, wherein the reverse transcriptase comprises a Moloney murine leukemia virus (MMLV)-type reverse transcriptase, wherein the MMLV adds three non-templated deoxycytidines (+CCC) to the 3′ end of the cDNA strand.

27. The method of claim 3, wherein the (+CCC) nucleotides anneal to complementary guanosine nucleotides at the 3′ end of the template switching oligonucleotide.

28. A kit comprising one or more of:
a first recognition agent; a streptavidin-conjugated second recognition agent; a biotinylated priming sequence comprising a second sequencing adapter; a transposase fused to protein A (pA-transposase) comprising a first sequencing adapter; a reverse transcriptase; an oligo(dT), and a random priming oligonucleotide, and all required reagents for template switching, each packaged in a separate container; a solid support; and instructions directing the method as recited in claim 1.
